SEQUENCE LISTING (6 sequences)

SEQ ID NO: 1 - protein, 2332 aa, Artificial Sequence
  synthetic
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 2 - protein, 1445 aa, Artificial Sequence
  synthetic
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 3 - protein, 20 aa, Artificial Sequence
  synthetic
SFSQNSRHPSQNPPVLKRHQ

SEQ ID NO: 4 - protein, 1702 aa, Artificial Sequence
  synthetic
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLYGGGSGGGSGGGSGGGSGGGSGGGSEPRGPTIKPCPPCKCPAPNAEGEPSVFIFPPKIKDVLMISLSPMVTCVVVDVSEDDPDVQISWFVNNVEVLTAQTQTHREDYNSTLRVVSALPIQHQDWMSGKEFKCKVNNKALPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 5 - protein, 2054 aa, Artificial Sequence
  synthetic
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLYGGGSGGGSGGGSGGGSGGGSGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 6 - protein, 21 aa, Artificial Sequence
  synthetic
SFSQNSRHPSQNPPVLKRHQR

(19) United States
(12) Patent Application Publication
(10) Pub. No.: US 2016/0000884 A1
(43) Pub. Date: Jan. 7, 2016

(54) Liquid Factor VIII Formulations

(71) Applicant: Novo Nordisk A/S, Bagsvaerd (DK)

(72) Inventors: Christian Rischel, Copenhagen S (DK); Hans Holmegaard Soerensen, Virum (DK); Michael Bech Jensen, Alleroed (DK); Thomas Bjerg, Copenhagen OE (DK)

(73) Assignee: NOVO NORDISK A/S, Bagsvaerd (DK)

(21) Appl. No.: 14/420,564

(22) PCT Filed: Aug. 13, 2013
(86) PCT No.: PCT/EP2013/066847

§ 371 Date: Feb. 9, 2015

Related U.S. Application Data
(60) Provisional application No. 61/692,744, filed on Aug. 24, 2012.
(60) Provisional application No. 61/782,743, filed on Mar. 14, 2013.

(30) Foreign Application Priority Data
Aug. 13, 2012 (EP) 12180239.1
Mar. 18, 2013 (EP) 13159832.8

(51) Int. Cl. A61K 38/37; C12Q 1/58; C12Q 1/56; A61K 38/38; C07K 19/00

(52) U.S. Cl. CPC A61K 38/37; A61K 38/38; C07K 19/00; C12Q 1/56; C12Q 1/58; A61K 2039/505

(57) ABSTRACT

The invention is directed to a liquid, aqueous formulation of coagulation Factor VIII, comprising a Factor VIII molecule, a calcium salt in a concentration of more than 10 mM, and a saccharide and/or polyol in a concentration of at least 100 mM, wherein the formulation has a pH from 5.5-7.5. The invention furthermore provides a method for optimising a liquid formulation of coagulation Factor VIII, the method comprising the steps of: (i) Providing one or more liquid formulations comprising Factor VIII to be tested; (ii) Adding a protein denaturant to said liquid formulations, and incubating the resulting solutions for a predetermined period of time; (iii) Analysing the incubated solutions of (ii) for the presence of dissociated Factor VIII; and (iv) Selecting one or more formulation(s) having a desired low level of dissociated Factor VIII.

### BACKGROUND

In subjects with a coagulopathy, such as in human beings with haemophilia A, various steps of the coagulation cascade are rendered dysfunctional due to, for example, the absence or insufficient presence of a coagulation factor. Such dysfunction of one part of the coagulation cascade results in insufficient blood coagulation and potentially life-threatening bleeding, or damage to internal organs, such as the joints. Individuals with haemophilia A may receive coagulation factor replacement therapy such as exogenous Factor VIII (FVIII).

All Factor VIII products currently on the market are supplied as freeze-dried preparations for intravenous infusion. Before use, the person performing the infusion must reconstitute the powder with liquid. This is time-consuming, can be a multi-step manual operation, and requires the user to visually monitor the dissolution, which can take several minutes and may cause some dose variability. A stable, liquid ready-to-use formulation of Factor VIII molecules (preferably in combination with a convenient delivery system) is therefore highly desirable. Liquid formulations with very high concentrations of stabilising excipients may give rise to local irritation and possibly phlebitis at the injection site due to the large difference in osmotic pressure between the injected liquid and the surrounding tissue. Liquid formulations with osmotic concentration close to physiological conditions are therefore particularly desirable.

Proteins have a large number of possible degradation pathways in aqueous solution. Liquid protein formulations must therefore be finely tuned in order to obtain stability during the shelf life required for practical use. A typical required shelf life is 2 years at storage temperatures of 2-8° C. in order to allow production, storage and distribution. In the process of discovering excipients, values of pH and other possible factors that may improve the stability in liquid formulation, it is impractical to only use the target storage conditions, since samples must be incubated for a very long time in order to observe the hypothesized stabilizing effect. Therefore, stability studies are often conducted under accelerated (stressed) conditions. These stressed conditions include, for example, high temperature, high humidity, intensive lighting, extreme pHs, increased air/water interfaces by vortexing or shaking, and/or repeated freeze/thaw cycles. Due to these stressed conditions a key issue is whether and how well the data from accelerated stability studies can be extrapolated to those under real-time conditions.

A method for conducting reliable stability studies avoiding long-term storage and avoiding stressed conditions is therefore highly desirable.

The stability of liquid formulations of Factor VIII has previously been described in the academic literature and in patents and patent applications.

Fatouros et al. (International Journal of Pharmaceutics 155, 121-131 (1997)) describe the influence of oxygen, metal ions, pH and ionic strength on the stability of B-domain deleted Factor VIII in liquid solution. Part of this work is also described in U.S. Pat. No. 5,962,650.

Fatouros et al. (Pharmaceutical Research 14, 1679-1684 (1997) and International Journal of Pharmaceutics 194, 69-79 (2000)) describe the stabilising effects of surfactants and in particular very high concentrations of carbohydrates. Part of this work is also described in U.S. Pat. No. 5,919,908.

WO2011/027152 describes liquid formulations of Factor VIII buffered with a combination of tris and potassium benzoate, containing EDTA and Calcium with a surplus of calcium relative to EDTA and additional excipients.

### SUMMARY

The present invention relates to a liquid pharmaceutical formulation of FVIII, which formulation may be used to treat a subject with haemophilia A.

The present inventors have now discovered that for Factor VIII molecules, including derivatives such as PEGylated Factor VIII molecules and others with protracted action, improved stability of liquid formulations at refrigerated temperatures is obtained by Calcium concentrations of above 10 mM, e.g. 15 mM or higher, in combination with concentrations of polyols of 100 mM or higher. Due to the highly stabilising effect of these concentrations of Calcium and certain polyols, stable liquid formulations with a low concentration of NaCl and a low osmotic concentration can be obtained.

Thus, in one aspect, the present invention is directed to liquid, aqueous formulations of coagulation Factor VIII, wherein the formulation comprises a Factor VIII molecule, one or more calcium salt(s) providing a concentration of calcium ions of more than 10 mM, and one or more polyols at a concentration of at least 100 mM, wherein the formulation has a pH in the interval 5.5-7.5.

In one aspect, the present invention is directed to liquid, aqueous formulations of coagulation Factor VIII, wherein the formulation comprises a Factor VIII molecule, a calcium salt in a concentration of at least 15 mM, and a saccharide and/or polyol in a concentration of at least 100 mM; wherein the formulation has a pH from 5.5-7.5.

The present inventors have now further discovered that the stabilizing effect of excipients on liquid formulations of multivalent protein may be accurately ascertained by accelerated assays in which chemical protein denaturants have been included and samples are incubated for a short time.

Thus, in another aspect, the present invention is directed to methods for optimising a liquid formulation of coagulation Factor VIII and identifying a stable liquid formulation of Factor VIII, the methods comprising the following steps: (i) Providing one or more liquid formulation(s) comprising FVIII and the one or more excipients that should be assessed for stabilising effect on FVIII; (ii) adding a selected protein denaturant (for example, guanidinium chloride or urea) to said liquid formulation(s) and incubating the resulting solution(s) for a predetermined period of time; (iii) analysing the incubated solutions of (ii) for the presence of dissociated Factor VIII; and (iv) selecting one or more formulation(s) having a desired low level of dissociated Factor VIII.

### DESCRIPTION

A stable liquid pharmaceutical formulation of an FVIII molecule of the present invention facilitates use of improved (ease of use) delivery systems. The stabilisation principle described can also be used to stabilise the Factor VIII molecules during manufacturing (up-stream purification steps, storage and handling).

### Coagulation Factor VIII Molecules

Factor VIII (FVIII) is a large, complex glycoprotein that is primarily produced by hepatocytes. FVIII has a size of approx. 300 kDa, including a signal peptide, and contains several distinct domains as defined by homology. There are three A-domains, a unique B-domain, and two C-domains. Small acidic regions C-terminal of the A1 (the a1 region) and A2 (the a2 region) and N-terminal of the A3 domain (the a3 region) play important roles in its interaction with other coagulation proteins, including thrombin and von Willebrand factor (vWF or VWF), the carrier protein for FVIII. The detailed domain structure can thus be listed as A1-a1-A2-a2-B-a3-A3-C1-C2.

FVIII circulates in plasma as two chains, separated at the B-a3-border, i.e. as an A1-a1-A2-a2-B/a3-A3-C1-C2 heterodimer. The protein structure is stabilised by bivalent metal ion-bindings. The A1-a1-A2-a2-B chain is termed the heavy chain (HC) while the a3-A3-C1-C2 chain is termed the light chain (LC).

Endogenous FVIII molecules circulate in vivo as a pool of molecules with B-domains of various sizes, the shortest having a C-terminal at position 740, i.e. at the C-terminal of A2-a2. These FVIII molecules with B-domains of different length all have full procoagulant activity. Upon activation with thrombin, FVIII is cleaved at the C-terminal of A1-a1 at position 372, at the C-terminal of A2-a2 at position 740 and between a3 and A3 at position 1689, the latter cleavage releasing the a3 region with concomitant loss of affinity for VWF. The thrombin-cleaved (activated) FVIII molecule is termed FVIIIa. The activation allows interaction of FVIIIa with phospholipid surfaces like activated platelets and activated factor IX (FIXa), i.e. the tenase complex is formed, allowing efficient activation of factor X (FX).

The terms “Factor VIII(a)” and “FVIII(a)” include both FVIII and FVIIIa.

“FVIII(a)” includes natural allelic variations of FVIII(a) that may exist and occur from one individual to another. FVIII(a) may be plasma-derived or recombinantly produced, using well known methods of production and purification. The degree and location of glycosylation, tyrosine sulfation and other post-translation modifications may vary, depending on the chosen host cell and its growth conditions.

Human FVIII consists of 2351 amino acids (including a signal peptide) and 2332 amino acids (without the signal peptide). The detailed domain structure, A1-a1-A2-a2-B-a3-A3-C1-C2 has the corresponding amino acid residues (referring to SEQ ID NO 1): A1 (1-336), a1 (337-372), A2 (373-710), a2 (711-740), B (741-1648), a3 (1649-1689), A3 (1690-2020), C1 (2021-2173) and C2 (2174-2332). “Native FVIII” is the human FVIII molecule derived from the full length sequence as shown in SEQ ID NO: 1 (amino acid 1-2332). The B-domain spans amino acids 741-1648 in SEQ ID NO 1.

SEQ ID NO: 1: Wild type human coagulation Factor
VIII
ATRRYYLGAVELSWDYMQSDLGELPVDARFPPRVPKSFPFNTSVVYKKTL
FVEFTDHLFNIAKPRPPWMGLLGPTIQAEVYDTVVITLKNMASHPVSLHA
VGVSYWKASEGAEYDDQTSQREKEDDKVFPGGSHTYVWQVLKENGPMASD
PLCLTYSYLSHVDLVKDLNSGLIGALLVCREGSLAKEKTQTLHKFILLFA
VFDEGKSWHSETKNSLMQDRDAASARAWPKMHTVNGYVNRSLPGLIGCHR
KSVYWHVIGMGTTPEVHSIFLEGHTFLVRNHRQASLEISPITFLTAQTLL
MDLGQFLLFCHISSHQHDGMEAYVKVDSCPEEPQLRMKNNEEAEDYDDDL
TDSEMDVVRFDDDNSPSFIQIRSVAKKHPKTWVHYIAAEEEDWDYAPLVL
APDDRSYKSQYLNNGPQRIGRKYKKVRFMAYTDETFKTREAIQHESGILG
PLLYGEVGDTLLIIFKNQASRPYNIYPHGITDVRPLYSRRLPKGVKHLKD
FPILPGEIFKYKWTVTVEDGPTKSDPRCLTRYYSSFVNMERDLASGLIGP
LLICYKESVDQRGNQIMSDKRNVILFSVFDENRSWYLTENIQRFLPNPAG
VQLEDPEFQASNIMHSINGYVFDSLQLSVCLHEVAYWYILSIGAQTDFLS
VFFSGYTFKHKMVYEDTLTLFPFSGETVFMSMENPGLWILGCHNSDFRNR
GMTALLKVSSCDKNTGDYYEDSYEDISAYLLSKNNAIEPRSFSQNSRHPS
TRQKQFNATTIPENDIEKTDPWFAHRTPMPKIQNVSSSDLLMLLRQSPTP
HGLSLSDLQEAKYETFSDDPSPGAIDSNNSLSEMTHFRPQLHHSGDMVFT
PESGLQLRLNEKLGTTAATELKKLDFKVSSTSNNLISTIPSDNLAAGTDN
TSSLGPPSMPVHYDSQLDTTLFGKKSSPLTESGGPLSLSEENNDSKLLES
GLMNSQESSWGKNVSSTESGRLFKGKRAHGPALLTKDNALFKVSISLLKT
NKTSNNSATNRKTHIDGPSLLIENSPSVWQNILESDTEFKKVTPLIHDRM
LMDKNATALRLNHMSNKTTSSKNMEMVQQKKEGPIPPDAQNPDMSFFKML
FLPESARWIQRTHGKNSLNSGQGPSPKQLVSLGPEKSVEGQNFLSEKNKV
VVGKGEFTKDVGLKEMVFPSSRNLFLTNLDNLHENNTHNQEKKIQEEIEK
KETLIQENVVLPQIHTVTGTKNFMKNLFLLSTRQNVEGSYDGAYAPVLQD
FRSLNDSTNRTKKHTAHFSKKGEEENLEGLGNQTKQIVEKYACTTRISPN
TSQQNFVTQRSKRALKQFRLPLEETELEKRIIVDDTSTQWSKNMKHLTPS
TLTQIDYNEKEKGAITQSPLSDCLTRSHSIPQANRSPLPIAKVSSFPSIR
PIYLTRVLFQDNSSHLPAASYRKKDSGVQESSHFLQGAKKNNLSLAILTL
EMTGDQREVGSLGTSATNSVTYKKVENTVLPKPDLPKTSGKVELLPKVHI
YQKDLFPTETSNGSPGHLDLVEGSLLQGTEGAIKWNEANRPGKVPFLRVA
TESSAKTPSKLLDPLAWDNHYGTQIPKEEWKSQEKSPEKTAFKKKDTILS
LNACESNHAIAAINEGQNKPEIEVTWAKQGRTERLCSQNPPVLKRHQREI
TRTTLQSDQEEIDYDDTISVEMKKEDFDIYDEDENQSPRSFQKKTRHYFI
AAVERLWDYGMSSSPHVLRNRAQSGSVPQFKKVVFQEFTDGSFTQPLYRG
ELNEHLGLLGPYIRAEVEDNIMVTFRNQASRPYSFYSSLISYEEDQRQGA
EPRKNFVKPNETKTYFWKVQHHMAPTKDEFDCKAWAYFSDVDLEKDVHSG
LIGPLLVCHTNTLNPAHGRQVTVQEFALFFTIFDETKSWYFTENMERNCR
APCNIQMEDPTFKENYRFHAINGYIMDTLPGLVMAQDQRIRWYLLSMGSN
ENIHSIHFSGHVFTVRKKEEYKMALYNLYPGVFETVEMLPSKAGIWRVEC
LIGEHLHAGMSTLFLVYSNKCQTPLGMASGHIRDFQITASGQYGQWAPKL
ARLHYSGSINAWSTKEPFSWIKVDLLAPMIIHGIKTQGARQKFSSLYISQ
FIIMYSLDGKKWQTYRGNSTGTLMVFFGNVDSSGIKHNIFNPPIIARYIR
LHPTHYSIRSTLRMELMGCDLNSCSMPLGMESKAISDAQITASSYFTNMF
ATWSPSKARLHLQGRSNAWRPQVNNPKEWLQVDFQKTMKVTGVTTQGVKS
LLTSMYVKEFLISSSQDGHQWTLFFQNGKVKVFQGNQDSFTPVVNSLDPP
LLTRYLRIHPQSWVHQIALRMEVLGCEAQDLY

The Factor VIII molecules included in the formulations of the present invention may also be B-domain-truncated/deleted FVIII molecules wherein the remaining domains correspond to the sequences as set forth in amino acid numbers 1-740 and 1649-2332 of SEQ ID NO: 1.

It follows that these FVIII molecules are recombinant molecules produced in transformed host cells, preferably of mammalian origin. However, the remaining domains (i.e. the three A-domains, the two C-domains and the a1, a2 and a3 regions) may differ slightly e.g. about 1%, 2%, 3%, 4% or 5% from the amino acid sequence as set forth in SEQ ID NO 1 (amino acids 1-740 and 1649-2332).

The FVIII molecules included in the formulation of the present invention may also be biologically active fragments of FVIII, i.e., FVIII wherein domain(s) other than the B-domain has/have been deleted or truncated, but wherein the FVIII molecule in the deleted/truncated form retains its ability to support the formation of a blood clot. FVIII activity can be assessed in vitro using techniques well known in the art. Examples of FVIII activity assays can be found in the Examples section.

Amino acid modifications (substitutions, deletions, etc.) may be introduced in the remaining domains, e.g., in order to modify the binding capacity of Factor VIII with various other components such as e.g. Von Willebrand Factor (vWF), low density lipoprotein receptor-related protein (LPR), various receptors, other coagulation factors, cell surfaces, etc. or in order to introduce and/or abolish glycosylation sites, etc. Other mutations that do not abolish FVIII activity may also be accommodated in a FVIII molecule/analogue for use in a formulation of the present invention.

The term “FVIII analogue” as used herein refers to a FVIII molecule (full-length or B-domain-truncated/deleted) wherein one or more amino acids have been substituted or deleted compared to SEQ ID NO 1 or, for B-domain truncated/deleted FVII molecules, the corresponding part of SEQ ID NO 1.

FVIII analogues also include FVIII molecules, in which one or more of the amino acid residues of the parent polypeptide have been deleted or substituted with other amino acid residues, and/or wherein additional amino acid residues has been added to the parent FVIII polypeptide.

Furthermore, the Factor VIII molecules/analogues may comprise other modifications in e.g. the truncated B-domain and/or in one or more of the other domains of the molecules (“FVIII derivatives”). These other modifications may be in the form of various molecules conjugated to the Factor VIII molecule, such as e.g. polymeric compounds, peptidic compounds, fatty acid derived compounds, etc.

The term “FVIII derivative” as used herein refers to a FVIII molecule (full-length or B-domain truncated/deleted) or a FVIII analogue, wherein one or more of the amino acids of the parent FVIII polypeptide have been chemically modified, e.g. by alkylation, PEGylation (including glycopegylation, wherein PEG is attached to one or more of the polypeptide's glycans, for example, as described in US 20100261872), acylation, ester formation or amide formation or the like to conjugate different functional groups to the FVIII polypeptide backbone, for instance protractive groups or half-life extending moieties. The term “FVIII derivative” also encompasses fusion proteins, wherein a FVIII molecule (full-length or B-domain truncated/deleted) is fused to another polypeptide, for instance albumin or an Fc domain or Fc derivative.

In the present context, the term “glycopegylated FVIII” is intended to designate a Factor VIII molecule (including full length FVIII and B-domain truncated/deleted FVIII) wherein one or more PEG group(s) has/have been attached to the FVIII polypeptide via the polysaccharide sidechain(s) (glycan(s)) of the polypeptide.

The terms “protractive groups”/“half-life extending moieties” is herein understood to refer to one or more chemical groups attached to one or more amino acid site chain functionalities such as —SH, —OH, —COOH, —CONH2, —NH2, or one or more N- and/or O-glycan structures and that can increase in vivo circulatory half-life of a number of therapeutic proteins/peptides when conjugated to these proteins/peptides.

Examples of protractive groups/half-life extending moieties include: Biocompatible fatty acids and derivatives thereof, Hydroxy Alkyl Starch (HAS) e.g. Hydroxy Ethyl Starch (HES), Poly(Glyₓ-Ser_{y})ₙ (Homo Amino acid Polymer (HAP)), Hyaluronic acid (HA), Heparosan polymers (HEP), Phosphorylcholine-based polymers (PC polymer), Fleximer® polymers (Mersana Therapeutics, MA, USA), Dextran, Poly-sialic acids (PSA), polyethylene glycol (PEG), an Fc domain, Transferrin, Albumin, Elastin like peptides, XTEN® polymers (Amunix, CA, USA), Albumin binding peptides, a von Willebrand factor fragment (vWF fragment), a Carboxyl Terminal Peptide (CTP peptide, Prolor Biotech, IL), and any combination thereof (see, for example, McCormick, C. L., A. B. Lowe, and N. Ayres, Water-Soluble Polymers, in Encyclopedia of Polymer Science and Technology. 2002, John Wiley & Sons, Inc.). The manner of derivatization is not critical and as can be elucidated from the above,

The term “Fc fusion protein” is herein meant to encompass FVIII fused to an Fc domain that can be derived from any antibody isotype. An IgG Fc domain will often be preferred due to the relatively long circulatory half-life of IgG antibodies. The Fc domain may furthermore be modified in order to modulate certain effector functions such as e.g. complement binding and/or binding to certain Fc receptors. Fusion of FVIII with an Fc domain, which has the capacity to bind to FcRn receptors, will generally result in a prolonged circulatory half-life of the fusion protein compared to the half-life of the wt FVIII.

It follows that a FVIII molecule for use in the present invention may also be a derivative of a FVIII analogue, such as, for example, a fusion protein of an FVIII analogue, a PEGylated or glycoPEGylated FVIII analogue, or a FVIII analogue conjugated to a heparosan polymer.

The term “FVIII variant” as used herein refers to the group of FVIII analogues and FVIII derivatives.

Examples of FVIII molecules for use in formulations of the present invention comprise for instance the FVIII molecules described in WO2010045568, WO2009062100, WO2010014708, WO2008082669, WO2007126808, US20100173831, US20100173830, US20100168391, US20100113365, US20100113364, WO200331464, WO2009108806, WO2010102886, WO2010115866, WO2011101242 (PCT/EP2011/051438), WO2011101284 (PCT/EP2011/051959), WO2011101277 (PCT/EP2011/051889), WO2011131510 (PCT/EP2011/055686), WO2012007324 (PCT/EP2011/061349), WO2011101267 (PCT/EP2011/051723), and WO2013083858.

Examples of FVIII molecules, which can be used in a formulation of the present invention include the active ingredient of Advate®, Helixate®, Kogenate®, Xyntha® as well as the FVIII molecule described in WO2008/135501 and WO2009/007451.

FVIII molecules included in a formulation of the present invention may also be FVIII derivatives or FVIII analogues or combinations thereof exhibiting substantially the same or improved biological activity relative to wild-type FVIII, when compared to human FVIII in a chromogenic assay (FVIII activity assay). Examples of FVIII activity assays can be found in the Examples section.

### Bioactivity

FVIII molecules included in the formulation according to the present invention are capable of functioning in the coagulation cascade in a manner that is functionally similar, or equivalent, to human FVIII, inducing the formation of FXa via interaction with FIXa on an activated platelet and supporting the formation of a blood clot. FVIII activity can be assessed in vitro using techniques well known in the art. Clot analyses, FX activation assays (often termed chromogenic assays), thrombin generation assays and whole blood thromboelastography are examples of such in vitro techniques. FVIII molecules for use in a formulation of the present invention may have FVIII activity that is at least about 10%, at least about 20%, at least about 30%, at least about 40%, at least about 50%, at least about 60%, at least about 70%, at least about 80%, at least about 90%, 100% or even more than 100% of that of native human FVIII when compared to human FVIII in a chromogenic assay (FVIII activity assay). Examples of FVIII activity assays can be found in the Examples section.

### B-Domain

The B-domain in FVIII spans amino acids 741-1648 of SEQ ID NO: 1. The B-domain is cleaved at several different sites, generating large heterogeneity in circulating plasma FVIII molecules. The exact function of the heavily glycosylated B domain is unknown. What is known is that the B-domain is dispensable for FVIII activity in the coagulation cascade. Recombinant FVIII is thus frequently produced in the form of B-domain-deleted/truncated molecules. The apparent lack of function is supported by the fact that B domain deleted/truncated FVIII appears to have in vivo properties identical to those seen for full length native FVIII. That being said there are indications that the B-domain may reduce the association with the cell membrane, at least under serum free conditions.

### B-Domain-Deleted/Truncated Factor VIII Molecules

Endogenous full length FVIII is synthesized as a single-chain precursor molecule. Prior to secretion, the precursor is cleaved into the heavy chain and the light chain. Recombinant B domain-deleted FVIII can be produced by means of two different strategies. Either the heavy chain without the B-domain and the light chain are synthesized individually as two different polypeptide chains (two-chain strategy) or the B domain-deleted FVIII is synthesized as a single precursor polypeptide chain (single-chain strategy) that is cleaved into the heavy and light chains in the same way as the full-length FVIII precursor.

In a B domain-deleted FVIII precursor polypeptide, produced by the single-chain strategy, the heavy and light chain moieties are often separated by a linker. To minimize the risk of introducing immunogenic epitopes in the B domain-deleted FVIII, the sequence of the linker may be derived from the FVIII B-domain. As a minimum, the linker must comprise a recognition site for the protease that cleaves the B domain-deleted FVIII precursor polypeptide into the heavy and light chain. In the B domain of full length FVIII, amino acid 1644-1648 constitutes this recognition site. The thrombin cleavage site leading to removal of the linker on activation of B domain-deleted FVIII is located in the heavy chain. Thus, the size and amino acid sequence of the linker is unlikely to influence its removal from the remaining FVIII molecule by thrombin activation. Deletion/truncation of the B domain is an advantage for production of FVIII. Nevertheless, parts of the B domain can be included in the linker without reducing the productivity. The negative effect of the B domain on productivity has not been attributed to any specific size or sequence of the B domain.

### Degradation

Factor VIII in liquid formulation is degraded by several mechanisms, including oxidation and dissociation between heavy and light chains. The dissociation of light- and heavy chain can be assessed in vitro by well-known techniques, e.g., by means of size-exclusion chromatography (SEC) as described in the Examples section.

Dissociation of Factor VIII molecules is observed in SEC as appearance of a peak with longer elution times than monomeric Factor VIII. This peak has been assigned to free Light Chain (Fatouros et al., International Journal of Pharmaceutics 155, pp 121, 1997). Thus, disscociation of FVIII molecules can be e.g. be assessed by assaying the level of free Light Chain.

### EMBODIMENTS

In one embodiment of the present invention the Factor VIII is recombinantly made full-length human FVIII. In another embodiment, the Factor VIII is a recombinantly made B-domain-truncated FVIII. In one embodiment of the invention, the Factor VIII is full-length human FVIII having the sequence as shown in SEQ ID NO: 1.

In one embodiment of the invention, the FVIII molecule is a FVIII sequence analogue; in another embodiment, the FVIII molecule is a FVIII sequence analogue exhibiting substantially the same or improved biological activity relative to wild-type FVIII when measured in a clot assay, e.g. as described in the Experimental section, below.

In one embodiment, the FVIII molecule is a B-domain truncated FVIII molecule produced by a vector encoding the amino acid sequence given in SEQ ID NO 2, which molecule comprises a 21 amino acid residue linker sequence (SFSQNSRHPSQNPPVLKRHQR) (SEQ ID NO 6).

SEQ ID NO 2:
ATRRYYLGAVELSWDYMQSDLGELPVDARFPPRVPKSFPFNTSWYKKTLF
VEFTDHLFNIAKPRPPWMGLLGPTIQAEVYDTVVITLKNMASHPVSLHAV
GVSYWKASEGAEYDDQTSQREKEDDKVFPGGSHTYVWQVLKENGPMASDP
LCLTYSYLSHVDLVKDLNSGLIGALLVCREGSLAKEKTQTLHKFILLFAV
FDEGKSWHSETKNSLMQDRDAASARAWPKMHTVNGYVNRSLPGLIGCHRK
SVYWHVIGMGTTPEVHSIFLEGHTFLVRNHRQASLEISPITFLTAQTLLM
DLGQFLLFCHISSHQHDGMEAYVKVDSCPEEPQLRMKNNEEAEDYDDDLT
DSEMDVVRFDDDNSPSFIQIRSVAKKHPKTWVHYIAAEEEDWDYAPLVLA
PDDRSYKSQYLNNGPQRIGRKYKKVRFMAYTDETFKTREAIQHESGILGP
LLYGEVGDTLLIIFKNQASRPYNIYPHGITDVRPLYSRRLPKGVKHLKDF
PILPGEIFKYKWTVTVEDGPTKSDPRCLTRYYSSFVNMERDLASGLIGPL
LICYKESVDQRGNQIMSDKRNVILFSVFDENRSWYLTENIQRFLPNPAGV
QLEDPEFQASNIMHSINGYVFDSLQLSVCLHEVAYWYILSIGAQTDFLSV
FFSGYTFKHKMVYEDTLTLFPFSGETVFMSMENPGLWILGCHNSDFRNRG
MTALLKVSSCDKNTGDYYEDSYEDISAYLLSKNNAIEPRSFSQNSRHPSQ
NPPVLKRHQREITRTTLQSDQEEIDYDDTISVEMKKEDFDIYDEDENQSP
RSFQKKTRHYFIAAVERLWDYGMSSSPHVLRNRAQSGSVPQFKKVVFQEF
TDGSFTQPLYRGELNEHLGLLGPYIRAEVEDNIMVTFRNQASRPYSFYSS
LISYEEDQRQGAEPRKNFVKPNETKTYFWKVQHHMAPTKDEFDCKAWAYF
SDVDLEKDVHSGLIGPLLVCHTNTLNPAHGRQVTVQEFALFFTIFDETKS
WYFTENMERNCRAPCNIQMEDPTFKENYRFHAINGYIMDTLPGLVMAQDQ
RIRWYLLSMGSNENIHSIHFSGHVFTVRKKEEYKMALYNLYPGVFETVEM
LPSKAGIWRVECLIGEHLHAGMSTLFLVYSNKCQTPLGMASGHIRDFQIT
ASGQYGQWAPKLARLHYSGSINAWSTKEPFSWIKVDLLAPMIIHGIKTQG
ARQKFSSLYISQFIIMYSLDGKKWQTYRGNSTGTLMVFFGNVDSSGIKHN
IFNPPIIARYIRLHPTHYSIRSTLRMELMGCDLNSCSMPLGMESKAISDA
QITASSYFTNMFATWSPSKARLHLQGRSNAWRPQVNNPKEWLQVDFQKTM
KVTGVTTQGVKSLLTSMYVKEFLISSSQDGHQWTLFFQNGKVKVFQGNQD
SFTPVVNSLDPPLLTRYLRIHPQSWVHQIALRMEVLGCEAQDLY

In one embodiment, the FVIII molecule is a two-chain B-domain truncated FVIII molecule consisting of a heavy chain-Linker sequence (A1-a1-A2-a2-L) and a light chain sequence (a3-A3-C1-C2) held together by non-covalent interactions. The Linker (L) is a 20 amino acid residue linker sequence (SFSQNSRHPSQNPPVLKRHQ) (SEQ ID NO 3); the heavy chain (A1-a1-A2-a2) and the light chain (a3-A3-C1-C2) correspond to the sequences as set forth in amino acid numbers 1-740 and 1649-2332, respectively, of SEQ ID NO: 1.

In one embodiment, the FVIII is attached to a protraction group. In a series of embodiments, the Factor VIII is (i) pegylated FVIII, (ii) a FVIII fusion protein, (iii) an albumin-fused FVIII, or (iv) an Fc region-fused FVIII.

In another embodiment, the Factor VIII is glycopegylated FVIII, such as glycopegylated recombinantly made full-length human FVIII or glycopegylated B-domain-truncated FVIII. In one embodiment, the FVII is attached to a polysaccharide (e.g., HEP, HES, HAS, PSA). In another embodiment, the Factor VIII is attached to polysialic acid (PSA) or heparosan (HEP).

In one embodiment, the FVIII molecule is a B-domain truncated FVIII molecule with a modified circulatory half-life, said molecule being covalently conjugated with a hydrophilic polymer via an O-linked oligosaccharide in the truncated B-domain, wherein FVIII activation results in removal of the covalently conjugated polymer. In different specific embodiments thereof, the hydrophilic polymer is polyethylene glycol (PEG), PSA, and HEP.

In one embodiment, the FVIII molecule is a B-domain truncated Factor VIII molecule with a modified circulatory half life, said molecule being covalently conjugated with a hydrophilic polymer via an O-linked oligosaccharide in the truncated B domain, wherein: (i) Factor VIII activation results in removal of the covalently conjugated hydrophilic polymer; and (ii) the heavy and light chain moieties of the FVIII precursor polypeptide are separated by a linker, wherein the sequence of the linker is derived from the FVIII B domain. In one embodiment, the length of the B domain is 20-30 amino acids. In one embodiment, the hydrophilic polymer is a polysaccharide; in one embodiment, said polysaccharide is PSA; in another embodiment, said polysaccharide is HEP. In one embodiment, the hydrophilic polymer is PEG. In different embodiments, the size of the PEG is from about 10,000 to about 160,000 Da or about 40,000 Da. In a series of embodiments, the PEG used has a size in the range of 2-160 kDa, or 2-80 kDa, or 5-80 kDa; or 5-60 kDa; or 10-80 kDa, or 10-60 kDa, or 20-60 kDa. In different embodiments, the PEG is a 2 kDa, 5 kDa, 10 kDa, 20 kDa, 40 kDa, or 80 kDa PEG. In one particular series of embodiments, the FVIII molecule attached to a protraction group as described above is a two-chain B-domain truncated FVIII molecule consisting of a heavy chain-Linker sequence (A1-a1-A2-a2-L) and a light chain sequence (a3-A3-C1-C2) held together by non-covalent interactions, wherein the Linker (L) is a 20 amino acid residue linker sequence (SFSQNSRHPSQNPPVLKRHQ) (SEQ ID NO 3); the heavy chain (A1-a1-A2-a2) and the light chain (a3-A3-C1-C2) correspond to the sequences as set forth in amino acid numbers 1-740 and 1649-2332, respectively, of SEQ ID NO: 1. In particular embodiments thereof, the described FVIII is attached to (i) one or more PEG group(s), (ii) one or more PSA group(s), (iii) one or more HEP group(s), or (iv) one or more protracting group(s), which is selected from PEG, PSA and HEP. In a further particular embodiment, the one or more protracting PEG/PSA/HEP group(s) has/have been attached to the FVIII polypeptide via the polysaccharide sidechain(s) (glycan(s)) of the polypeptide; in a further embodiment, said protracting group(s) has/have been attached to the FVIII polypeptide via a glycan located within the linker sequence (SEQ ID 3). In further embodiments, the PEG group is a 20-60 kDa PEG or a 40 kDa PEG.

In one embodiment, the FVIII in the formulation of the present invention is

a two-chain B-domain truncated FVIII molecule consisting of a heavy chain-Linker sequence (A1-a1-A2-a2-L) and a light chain sequence (a3-A3-C1-C2) held together by non-covalent interactions, wherein the Linker (L) is a 20 amino acid residue linker sequence (SFSQNSRHPSQNPPVLKRHQ) (SEQ ID NO 3); the heavy chain (A1-a1-A2-a2) and the light chain (a3-A3-C1-C2) correspond to the sequences as set forth in amino acid numbers 1-740 and 1649-2332, respectively, of SEQ ID NO: 1, wherein one or more PEG group(s) has/have been attached to the FVIII polypeptide via a glycan located within the linker sequence (SEQ ID 3). In further embodiments, the PEG group is a 20-60 kDa PEG or a 40 kDa PEG.

In one embodiment, the FVIII in the formulation of the present invention is

a two-chain B-domain truncated FVIII molecule consisting of a heavy chain-Linker sequence (A1-a1-A2-a2-L) and a light chain sequence (a3-A3-C1-C2) held together by non-covalent interactions, wherein the Linker (L) is a 20 amino acid residue linker sequence (SFSQNSRHPSQNPPVLKRHQ) (SEQ ID NO 3); the heavy chain (A1-a1-A2-a2) and the light chain (a3-A3-C1-C2) correspond to the sequences as set forth in amino acid numbers 1-740 and 1649-2332, respectively, of SEQ ID NO: 1, wherein one or more HEP group(s) has/have been attached to the FVIII polypeptide via a glycan located within the linker sequence (SEQ ID 3).

In one embodiment, the FVIII in the formulation of the present invention is

a two-chain B-domain truncated FVIII molecule consisting of a heavy chain-Linker sequence (A1-a1-A2-a2-L) and a light chain sequence (a3-A3-C1-C2) held together by non-covalent interactions, wherein the Linker (L) is a 20 amino acid residue linker sequence (SFSQNSRHPSQNPPVLKRHQ) (SEQ ID NO 3); the heavy chain (A1-a1-A2-a2) and the light chain (a3-A3-C1-C2) correspond to the sequences as set forth in amino acid numbers 1-740 and 1649-2332, respectively, of SEQ ID NO: 1, wherein one or more PSA group(s) has/have been attached to the FVIII polypeptide via a glycan located within the linker sequence (SEQ ID 3).

In one embodiment, the FVIII in the formulation of the present invention is a B-domain truncated FVIII molecule given in SEQ ID NO 2, wherein one or more PEG group(s) has/have been attached to the FVIII polypeptide via a glycan located within the linker sequence (SEQ ID 3). In further embodiments, the PEG group is a 20-60 kDa PEG or a 40 kDa PEG.

In one embodiment, the FVIII in the formulation of the present invention is a B-domain truncated FVIII molecule given in SEQ ID NO 2, wherein one or more HEP group(s) has/have been attached to the FVIII polypeptide via a glycan located within the linker sequence (SEQ ID 3).

In one embodiment, the FVIII in the formulation of the present invention is a B-domain truncated FVIII molecule given in SEQ ID NO 2, wherein one or more PSA group(s) has/have been attached to the FVIII polypeptide via a glycan located within the linker sequence (SEQ ID 3).

### Aqueous Formulations

The concentration of Factor VIII in the formulation of the present invention is typically in the range of 10-10.000 IU/mL. In different embodiments, the concentration of FVIII molecule in the formulations of the invention is in the range of 10-8000 IU/mL, or 10-6000 IU/mL, or 10-4000 IU/mL, or 10-2500 IU/mL, or 30-4000 IU/mL, or 30-2500 IU/mL, or 50-2500 IU/mL, or 50-1250 IU/mL, or 100-2500 IU/mL.

One IU (International Unit) is defined as the amount of FVIII found in 1 mL of fresh, pooled normal human plasma.

In one embodiment of the invention the pharmaceutical formulation is an aqueous solution. The term “aqueous formulation” is defined as a formulation comprising at least 50% w/w water. Likewise, the term “aqueous solution” is defined as a solution comprising at least 50% w/w water.

### Salts

The formulation according to the present invention comprises a calcium salt. The formulation may also contain a sodium salt.

In one embodiment the formulation contains at least 15 mM of a calcium salt. In one series of embodiments, the formulation comprises 15-100 mM of a calcium salt, or 15-80 mM or 15-60 mM or 15-45 mM or 15-30 mM or 15-25 mM or 15-20 mM; or at least 20 mM of a calcium salt, or 20-100 mM, or 20-80 mM, or 20-60 mM, or 20-45 mM, or 20-45 mM, or 20-40 mM, or 20-30 mM, or 25-35 mM, or at least 30 mM, or 30-45 mM, or about 30 mM. In one particular embodiment, the formulation contains 25-35 mM of a calcium salt.

The calcium salt may, for example, be selected from the group of calcium chloride, calcium acetate, calcium lactate, calcium benzoate and mixtures thereof, and other soluble calcium salts well known by the person skilled in the art. In one embodiment, the calcium salt is calcium chloride.

In one embodiment, the formulation does not contain EDTA.

In one embodiment the formulation contains at least 5 mM of a sodium salt. In one series of embodiments, the formulation comprises 5-500 mM of a sodium salt, or 15-150 mM or 15-125 mM or 15-100 mM; or at least 20 mM of a calcium salt, or 20-150 mM or 20-130 mM or 20-100 mM; or at least 30 mM, or 30-150 mM or 50-150 mM. In one embodiment, the concentration of the sodium salt is 100 mM, or 5-100 mM, or 50-100 mM, or below 50 mM, or 5-50 mM.

Sodium salt(s) used for pH adjustment, typically in the form of NaOH, is/are included in the specified Sodium concentrations.

The sodium salt may, for example, be selected from the group of sodium chloride, sodium acetate, sodium lactate, sodium benzoate, and mixtures thereof, and other soluble sodium salts well known by the person skilled in the art

In one embodiment of the invention, the sodium salt is sodium chloride; in another embodiment, the salt is sodium acetate. In a third embodiment, the formulation of the invention contains a mixture of sodium chloride and sodium acetate.

### Buffers

The formulation according to the invention may comprise a buffering system. The buffer (or buffering substance) may be selected from the group consisting of acetate, benzoate, carbonate, citrate, glycylglycine, histidine or derivatives of histidine, Hepes, glycine, phosphate, hydrogen phosphate, and tris(hydroxymethyl)-aminomethan (TRIS), bicine, tricine, succinate, aspartic acid, glutamic acid or mixtures thereof. In one embodiment of the invention, the concentration of the buffering substance is 1-100 mM, such as, e.g., 1-50 mM or 1-25 mM or 1-20 mM or 5-20 mM or 5-15 mM.

In one embodiment of the invention the formulation comprises histidine, preferably L-histidine. In one embodiment thereof, the concentration of histidine is 1-100 mM, such as, e.g., 1-50 mM or 1-25 mM or 1-20 mM or 5-20 mM or 5-15 mM.

The liquid formulation of the invention typically has a pH from 5.5 to 7.5. In different embodiments, the formulation has a pH of 6.0-7.0 or 6.2-6.8 or 6.3-6.7

Saccharides and/or Polyols

The formulation of the invention further comprises a saccharide (sugar) and/or a polyol (sugar alcohol). The saccharide may, for example, be selected from the group of mono-, di-, or polysaccharides, and water-soluble glucans (including for example the monosaccharides fructose, glucose, mannose, the disaccharides lactose, sucrose, trehalose, and the polysaccharides dextran, raffinose, stachyose). The polyol may, for example, be selected from the group of sugar alcohols (including for example, mannitol, sorbitol, inositol, galactitol, dulcitol, xylitol, and arabitol), alditols (e.g. glycerol (glycerine), 1,2-propanediol (propyleneglycol), 1,3-propanediol, 1,3-butanediol), polyethyleneglycol, or mixtures thereof.

If more than one saccharide and/or polyol are included in the formulation, the described concentrations are meant to designate the total amount of “saccharide and/or polyol” present in the formulation.

In one embodiment of the invention, the FVIII formulation comprises one or more saccharides and/or sugar alcohols from the group of: sucrose, sorbitol, glycerol, raffinose, stachyose, mannitol, sorbitol, or mixtures thereof.

In one embodiment, the FVIII formulation comprises a saccharide and/or sugar alcohol in a concentration of at least 200 mM.

In one embodiment the FVIII formulations according to the invention comprise one or more saccharide(s) and do not comprise a polyol. In another embodiment, the formulations comprise a single saccharide component and do not comprise a polyol. In specific embodiments of the above, the saccharide is sucrose.

In one embodiment, the formulations comprise one or more polyol(s) and do not comprise a saccharide. In another embodiment, the formulations comprise a single polyol component and do not comprise a saccharide. In specific embodiments of the above, the saccharide is sorbitol or mannitol.

In one embodiment of the invention, the formulation comprises saccharide and/or sugar alcohol in concentrations leading to a calculated osmotic concentration (“X”) of the formulation of equal to or below 1.50 Osm/L. In one embodiment of the invention, the formulation thus comprises a saccharide and/or sugar alcohol in a concentration of from 100 mM to a concentration of X mM, wherein X is defined as the value (mM) for which the calculated osmotic concentration of the formulation reaches 1.50 Osm/L. In another one embodiment, the formulation comprises a saccharide and/or sugar alcohol in a concentration of from 200 mM to a concentration of X mM, wherein X is defined as the value (mM) for which the calculated osmotic concentration of the formulation reaches 1.50 Osm/L.

When calculating the osmotic concentration of a given formulation, and thereby determining the value a X for the formulation in question, all components in the formulation is included in the calculation (e.g., CaCl₂, NaCl, buffering substance, methionine). Calculation of osmotic concentration is described in the present application (see section, “Osmotic concentration”, below). However, the theoretical calculation of osmotic concentration is well known to the person skilled in the art.

In one embodiment of the invention, the formulation comprises a saccharide and/or sugar alcohol in a concentration of at least 100 mM, or at least 200 mM, or 100-1800 mM, or 300-1800 mM, or 100-1500 mM, or 200-1800 mM, or 200-1500 mM, or 100-1000 mM, or 200-1000 mM, or 300-1000 mM, or 200-800 mM, or 300-800 mM, or 400-800 mM, or 500-800 mM, or 500-700 mM.

In one embodiment, the formulation of the invention comprises sucrose. In one embodiment, the concentration of sucrose is 100-1000 mM, or 150-1750 mM, or 200-1000 mM, or 300-1000 mM, or 200-800 mM, or 300-800 mM, or 440-730 mM, or 400-800 mM, or 500-800 mM, or 500-700 mM; in another embodiment, the concentration of sucrose is 50-600 mg/mL, or 100-600 mg/mL, or 100-450 mg/mL or 150-450 mg/mL or 150-250 mg/mL (100 mg/mL of sucrose corresponding to 292 mM).

In another embodiment, the formulation of the invention comprises sorbitol. In different embodiments, the concentration of sorbitol is at least 400 mM, or 400-1500 mM, or 100-800 mg/mL, or 100-650 mg/mL, or 150-650 mg/mL, or 150-500 mg/mL, or 150-250 mg/mL (100 mg/mL of sorbitol corresponding to 549 mM)

### Other Excipients

The formulation of the present invention may further contain additional excipients. Examples of standard excipients for use in a pharmaceutical formulation according the present invention are preservative(s), antioxidants(s), and surfactant(s).

In one embodiment of the invention a reducing agent such as methionine (or other sulphuric amino acids or sulphuric amino acid analogues) may be added to inhibit oxidation of methionine residues to methionine sulfoxide. By “inhibit” is intended minimal accumulation of methionine-oxidized species during manufacturing and over time. Inhibiting methionine oxidation results in greater retention of the polypeptide in its proper molecular form. The amount to be added should be an amount sufficient to inhibit oxidation of the methionine residues such that the amount of methionine sulfoxide is acceptable to regulatory agencies. Typically, this means that the formulation contains no more than about 10% to about 30% methionine sulfoxide. Generally, this can be achieved by adding methionine such that the ratio of methionine added to methionine residues of Factor VIII is at least about 1:1

In one embodiment of the invention, the formulation comprises methionine, e.g., L-methionine. In one embodiment thereof, the concentration of the methionine is 0.05-100 mM, such as, e.g., 0.1-10 mM or 0.1-2 mM or 0.2-0.5 mM.

In one embodiment of the invention the formulation further comprises a surfactant. Typical surfactants (with examples of trade names given in brackets [ ]) are polyoxyethylene sorbitan fatty acid esters such as polyoxyethylene (20) sorbitan monolaurate [Tween 20], polyoxyethylene (20) sorbitan monopalmitate [Tween 40] or polyoxyethylene (20) sorbitan monooleate [Tween 80], poloxamers such as polyoxypropylene-polyoxyethylene block copolymer [Pluronic F68/poloxamer 188], polyethylene glycol octylphenyl ether [Triton X-100] or polyoxyethyleneglycol dodecyl ether [Brij 35]. The use of a surfactant in pharmaceutical formulations is well-known to the skilled person. For convenience reference is made to Remington: The Science and Practice of Pharmacy, 19th edition, 1995.

In one embodiment of the invention, the formulation comprises sorbitan monooleate [Tween 80]. In one embodiment thereof, the concentration of the sorbitan monooleate [Tween 80] is 0.01-0.5 mg/mL, such as, e.g., 0.05-0.3 mg/mL or 0.05-0.2 mg/mL, or about 0.1 mg/mL.

In one embodiment of the invention, the formulation comprises poloxamer 188. In one embodiment thereof, the concentration of poloxamer 188 is 0.01-5 mg/mL, such as, e.g., 0.05-3 mg/mL or 0.25-2 mg/mL, or about 0.5 mg/mL.

In one embodiment of the invention, the formulation contains FVIII, L-histidine, Tween® 80, L-methionine, NaCl, sucrose, and CaCl₂; in another embodiment of the invention, the formulation contains FVIII, 10 mM L-histidine, 0.1 mg/ml Tween® 80, 0.37 mM L-methionine, 78 mM NaCl, 188 mg/ml sucrose, and 30 mM CaCl₂, pH 6.7; in another embodiment, the formulation comprises FVIII, 20-40 mM CaCl₂, 500-800 mM sucrose or 150-250 mg/mL sorbitol. In particular embodiments of either of these embodiments, said FVIII is:

(i) a FVIII molecule comprising the domains corresponding to the sequences as set forth in amino acid numbers 1-740 and 1649-2332 of SEQ ID NO 1; or
(ii) a B-domain truncated FVIII molecule given in SEQ ID NO 2; or
(ii) a two-chain B-domain truncated FVIII molecule consisting of a heavy chain-Linker sequence (A1-a1-A2-a2-L) and a light chain sequence (a3-A3-C1-C2) held together by non-covalent interactions. The Linker (L) is a 20 amino acid residue linker sequence (SFSQNSRHPSQNPPVLKRHQ) (SEQ ID NO 3), the heavy chain (A1-a1-A2-a2) and the light chain (a3-A3-C1-C2) corresponding to the sequences as set forth in amino acid numbers 1-740 and 1649-2332, respectively, of SEQ ID NO 1; or
(iv) a B-domain truncated FVIII molecule given in SEQ ID NO 2, wherein one or more PEG group(s) has/have been attached to the FVIII polypeptide via a glycan located within the linker sequence (SEQ ID 3); or
(v) a two-chain B-domain truncated FVIII molecule consisting of a heavy chain-Linker sequence (A1-a1-A2-a2-L) and a light chain sequence (a3-A3-C1-C2) held together by non-covalent interactions, wherein the Linker (L) is a 20 amino acid residue linker sequence (SFSQNSRHPSQNPPVLKRHQ) (SEQ ID NO 3); the heavy chain (A1-a1-A2-a2) and the light chain (a3-A3-C1-C2) correspond to the sequences as set forth in amino acid numbers 1-740 and 1649-2332, respectively, of SEQ ID NO: 1, wherein one or more PEG group(s) has/have been attached to the FVIII polypeptide via a glycan located within the linker sequence (SEQ ID 3); or
(vi) a B-domain truncated FVIII molecule with a modified circulatory half-life, said molecule being covalently conjugated with a hydrophilic polymer via an O-linked oligosaccharide in the truncated B-domain, wherein FVIII activation results in removal of the covalently conjugated polymer. In different specific embodiments thereof, the hydrophilic polymer is polyethylene glycol (PEG), PSA, and HEP;
(vii) a B-domain truncated Factor VIII molecule with a modified circulatory half life, said molecule being covalently conjugated with a hydrophilic polymer via an O-linked oligosaccharide in the truncated B domain, wherein: (i) Factor VIII activation results in removal of the covalently conjugated hydrophilic polymer; and (ii) the heavy and light chain moieties of the FVIII precursor polypeptide are separated by a linker, wherein the sequence of the linker is derived from the FVIII B domain. In one embodiment, the length of the B domain is 20-30 amino acids; or
(vi) the active ingredient of Advate®, or
(vii) the active ingredient of Helixate®; or
(viii) the active ingredient of Kogenate®, or
(ix) the active ingredient of Xyntha®, or
(x) a FVIII molecule manufactured as described by Thim L. et al. (Haemophilia 2010; 16:349-359); or
(xi) a FVIII molecule manufactured as described in WO 2009108806,

### Antioxidation

An antioxidant effect can be achieved by displacing oxygen (air) from contact with the product. In particular embodiments, the formulation does not include an antioxidant; instead the susceptibility of the FVIII to oxidation is controlled by exclusion of atmospheric air or by displacing oxygen (air) from contact with the product. This may e.g. be accomplished by saturating the liquid formulation with either nitrogen, helium or argon and sealing the final container after displacing the air above the product with the gas. The displacement of oxygen (air) may e.g. be carried out as a “degassing” process where the formulation is subjected to one or more cycles of (i) exposure to an inert gas (argon, helium or nitrogen) and/or (ii) evacuation of the chamber containing the formulation to a pressure below atmospheric pressure. In one particular embodiment, the formulation is sterile filtered, distributed in vials, and degassed by three cycles of exposure to pure N₂, interspersed by brief evacuation of the chamber to 0.1 bar pressure, and the vials are then sealed with pure N₂ in the headspace.

The use of an antioxidant may of course also be combined with the exclusion of atmospheric air. Furthermore, the formulation may be protected from light; said protection may of course be combined with either or both of exclusion of atmospheric air and the use of an antioxidant.

Thus, the present invention also provides an air-tight container (e.g. a vial or a cartridge (such as a cartridge for a pen applicator)) containing a liquid, aqueous pharmaceutical formulation as defined herein, and optionally an inert gas. The inert gas may be selected from the group consisting of nitrogen, helium or argon. In the present context, the term “air-tight container” means a container having a low permeability to oxygen (air). The container (e.g. vial or cartridge or syringe) is typically made of glass or plastic, in particular glass, optionally closed by a rubber septum or other closure means allowing for penetration with preservation of the integrity of the pharmaceutical formulation. In a further embodiment, the container is a vial or cartridge enclosed in a sealed bag, e.g. a sealed plastic bag, such as a laminated (e.g. metal (such as aluminium) laminated plastic bag).

### Administration and Treatment

In one embodiment of the invention the formulations are pharmaceutical formulations intended for administration to a subject. The formulations are typically administered by parenteral administration, which may be performed by subcutaneous, intramuscular, intraperitoneal or intravenous injection by means of a syringe, optionally a pen-like syringe. Alternatively, parenteral administration can be performed by means of an infusion pump.

The present invention also encompasses a method of treating haemophilia A, which method comprises administering a formulation according to the present invention to a subject in need thereof.

The term “subject”, as used herein, includes any human patient, or non-human vertebrate.

The term “treating” or “treatment”, as used herein, refers to the medical therapy of any human or other vertebrate subject in need thereof. Said subject is expected to have undergone physical examination by a medical practitioner, or a veterinary medical practitioner, who has given a tentative or definitive diagnosis which would indicate that the use of said specific treatment is beneficial to the health of said human or other vertebrate. The timing and purpose of said treatment may vary from one individual to another, according to the status quo of the subject's health. Thus, said treatment may be prophylactic, palliative, symptomatic and/or curative. In terms of the present invention, prophylactic, palliative, symptomatic and/or curative treatments may represent separate aspects of the invention.

Said haemophilia A may be severe, moderate or mild. The clinical severity of haemophilia A is determined by the concentration of functional units of FVIII in the blood and is classified as mild, moderate, or severe. Severe haemophilia is defined by a clotting factor level of <0.01 U/ml corresponding to <1% of the normal level, while moderate and mild patients have levels from 1-5% and >5%, respectively.

### Osmotic Concentration

Osmotic concentration, formerly known as osmolarity, is the measure of solute concentration, defined as the number of osmoles (Osm) of solute per litre (L) of solution (osmol/L or Osm/L). Whereas molarity measures the number of moles of solute per unit volume of solution, osmolarity measures the number of osmoles of solute particles per unit volume of solution. Osmolality is a measure of the osmoles of solute per kilogram of solvent (osmol/kg or Osm/kg). Molarity and osmolarity are in theory temperature dependent. This is because water changes its volume with temperature. However, if the concentration of solutes is low, osmolarity and osmolality are considered equivalent.

The theoretical calculation of osmotic concentration is well known to the person skilled in the art. Briefly, one calculates for each component of the solution the product of the osmotic coefficient f, the number of particles n into which the molecule dissociates in water, and the molar concentration, and sums the result over all components.

Thus, the osmotic concentration of a solution can be calculated from the following expression: Osm/L=Σᵢfᵢ nᵢ Cᵢ, where the index i represents the identity of a particular component; fᵢ is the osmotic coefficient for a particular component; n is the number of particles into which the molecule dissociates in water; C is the molar concentration of the component. As previously mentioned, the molar concentration has a slight temperature dependence; for the present purpose we refer to the concentration at 25° C.

An alternative way to assess the osmotic pressure that a solution may exert after injection is by evaluating the osmolality, in which the content of the components is evaluated relative to solvent mass. Osmolality and osmotic concentration can easily be interconverted if the density of the solution and the dry mass of the dissolved components are known. Osmolality can be measured by a number of methods, most commonly freezing point depression.

For example, for water, 1 Osmol of a solute added to 1 kg of water lowers the freezing point by 1.86° C. Methods for measuring the osmolality of a solution by freezing point depression are described, for example, in the European Pharmacopeia 2.2.35 and the U.S. Pharmacopeia chapter 785.

The table below lists the osmotic coefficients and number of particles n for some important excipients. For other components, a value of f=1 provides a sufficiently good approximation for practical purposes, and the value of n is well known for essentially all compounds relevant for pharmaceutical preparations for parenteral use.

| Excipient | f | n |
|---|---|---|
| NaCl | 0.93 | 2 |
| CaCl₂ | 0.86 | 3 |
| Na acetate | 0.95 | 2 |
| Sucrose | 1.02 | 1 |
| Glycerol | 1.01 | 1 |
| Histidine | 1.0 | 1 |
| L-Methionine | 1.0 | 1 |
| Poloxamer-188 | 1.0 | 1 |
| Polysorbate 80 | 1.0 | 1 |

In different embodiments of the invention, the formulation has a calculated osmotic concentration of below 1.50 Osm/L, below 1.20 Osm/L, below 1.00 Osm/L, or below 0.90 Osm/L.

### Accelerated Assays for Ascertaining Stabilising Effect of Excipients in a FVIII Formulation

The present inventors have now further discovered that the stabilizing effect of excipients on liquid formulations of multivalent protein may be accurately ascertained by accelerated assays in which chemical protein denaturants have been included and samples are incubated for a short time.

Thus, incubation of liquid formulations of Factor VIII molecules (including analogues and derivatives) with a protein denaturant followed by analysis (e.g. by size-exclusion chromatography (SEC) as described in the Examples section) is a useful tool for rapidly investigating formulations. The samples may also be analysed for activity by e.g. chromogenic assay as described in the Examples section. The method according to the present invention provides a rapid and reliable way of doing accelerated stability studies. The method provides a way of avoiding long term storage of samples and/or avoiding subjecting the tested samples to stressed conditions. Stressed conditions may render the obtained results difficult to extrapolate to those under real-time conditions. The method according to the present invention provides a way of rapidly and accurately identifying a sample of FVIII formulations having a low formation of free Light chain. i.e., providing a way of rapidly and accurately identifying a stable formulation.

Where it takes months or even years to obtain real-time stability data for a given formulation, the present method provides data with a short period of time, typically within a week or less, typically even within 24-48 hours.

Chemical protein denaturants are characterized by a destabilization of the protein structure due to the composition of the solution, rather than due to external stress such as extreme temperature, mechanical stress or light. Examples of chemical protein denaturants are chaotropic agents such as guanidinium chloride, urea, thiourea, ethanol and other compounds well known to the person skilled in the art. Conditions of pH below 5.5 or above 8.0 can also serve as chemical denaturants for Factor VIII.

In one embodiment of the invention, the denaturant is guanidinium chloride (GuHCl). In another embodiment, the denaturant is urea. In one series of embodiments, the denaturant is guanidinium chloride in a concentration of 0.1-1.0 M, such as, e.g., 0.2-0.8 M or 0.2 M or 0.4M. In another series of embodiments, the denaturant is urea in a concentration of 1-5 M, such as, e.g., 1-3 M or 1-2 M.

The formulations may be analysed by any method that probes the presence of intact Factor VIII molecules. Particularly suited are chromatographic methods which provide separate signals for the intact Factor VIII molecules and for either the dissociated light chain or the dissociated heavy chain, or separate signals for all three.

In one embodiment, the formulations are analysed by size-exclusion chromatography. In another embodiment, the formulations are analysed by Field Flow Fractionation. In another embodiment, the formulations are analysed by ion-exchange chromatography. In another embodiment, the formulations are analysed by hydrophobic interaction chromatography. In another embodiment, the formulations are analysed by analytical ultracentrifugation. Other separation methods well known to the person skilled in the art may be similarly used.

### Incubation Time and Temperature:

After addition of the denaturant to the FVIII formulations, the denaturant-containing formulations are typically incubated at about 5° C. for at least 1 hour, more preferred for 24 hours or longer. In different embodiments, the incubation time is 12-240 hours, 12-120 hours, or 24-120 hours, or 24-60 hours

### LIST OF EMBODIMENTS

A number of different embodiments of the invention are mentioned in the following:

### Embodiment 1

A liquid, aqueous formulation of coagulation Factor VIII, comprising a Factor VIII polypeptide, a calcium salt in a concentration of at least 15 mM, a sodium salt in a concentration of at least 10 mM; wherein the formulation has a pH from 6.0-7.5.

### Embodiment 2

The formulation of Embodiment 1, comprising calcium salt in a concentration of 20-45 mM.

### Embodiment 3

The formulation according to Embodiment 1 or Embodiment 2, wherein the salt is calcium chloride.

### Embodiment 4

The formulation according to any one of Embodiments 1-3, wherein the concentration of the sodium salt is at most 100 mM.

### Embodiment 5

The formulation according to any one of Embodiments 1-4, where the sodium salt is sodium chloride or sodium acetate.

### Embodiment 6

The formulation according to any one of Embodiments 1-5, further containing a saccharide or sugar alcohol in a concentration of at least 200 mM.

### Embodiment 7

The formulation according to Embodiment 6, wherein the formulation contains sucrose in a concentration of at least 200 mM and at most 800 mM.

### Embodiment 8

The formulation according to Embodiment 6, wherein the formulation contains sorbitol in a concentration of at least 400 mM.

### Embodiment 9

The formulation according to any one of Embodiments 1-8, wherein the Factor VIII polypeptide is recombinant full length FVIII or a recombinant B-domain truncated FVIII.

### Embodiment 10

The formulation according to Embodiment 9, wherein the Factor VIII polypeptide is attached to a protraction group

### Embodiment 11

The formulation according to Embodiment 10, wherein the Factor VIII polypeptide is a pegylated FVIII, or an albumin-fused FVIII, or an Fc region-fused FVIII.

### Embodiment 12

The formulation according to Embodiment 10, wherein the Factor VIII polypeptide is a glycopegylated B-domain truncated FVIII.

### Embodiment 13

The formulation according to any one of Embodiments 1-12, having a pH from 6.0 to 7.0, or from 6.4 to 7.0.

### Embodiment 14

A method for optimising a liquid formulation of coagulation Factor VIII, the method comprising the steps of:

(i) Providing a variety of liquid formulations comprising Factor VIII to be tested;
(ii) Adding a protein denaturant to said liquid formulations, and incubating the resulting solutions for a predetermined period of time;
(iii) Analysing the incubated solutions of (ii) for the presence of free FVIII light chain;
(iv) Selecting one or more formulation(s) having a desired low level of free light chain.

### Embodiment 15

A method for identifying a stable liquid formulation of Factor VIII, the method comprising the steps of:

(i) Providing a variety of liquid formulations comprising Factor VIII to be tested;
(ii) Adding a protein denaturant to said liquid formulations, and incubating the resulting solutions for a predetermined period of time;
(iii) Analysing the incubated solutions of (ii) for the presence of free FVIII light chain;
(iv) Selecting one or more formulation(s) having a desired low level of free light chain.

### Embodiment 16

The method according to Embodiment 14 or Embodiment 15, wherein the protein denaturant is guadinium chloride or urea.

### Embodiment 17

The method according to any one of Embodiments 14-16, wherein the Factor VIII polypeptide is recombinant full length FVIII or a recombinant B-domain truncated FVIII.

### Embodiment 18

The method according to Embodiment 17, wherein the Factor VIII polypeptide is attached to a protraction group.

### Embodiment 19

The method according to Embodiment 18, wherein the Factor VIII polypeptide is a pegylated FVIII, or an albumin-fused FVIII, or an Fc region-fused FVIII.

### Embodiment 20

The method according to Embodiment 19, wherein the Factor VIII polypeptide is a glycopegylated B-domain truncated FVIII.

Further embodiments are:

### Embodiment 21

A liquid, aqueous formulation of coagulation Factor VIII, comprising a Factor VIII polypeptide, a calcium salt in a concentration of at least 15 mM, and a polyol in a concentration of at least 100 mM, wherein the formulation has a pH from 5.5-7.0

### Embodiment 22

The formulation of Embodiment 21, comprising calcium salt in a concentration of 15-100 mM, or 20-45 mM.

### Embodiment 23

The formulation according to Embodiment 21 or Embodiment 22, wherein the salt is calcium chloride.

### Embodiment 24

The formulation according to any one of Embodiments 21-23, wherein the calculated osmotic concentration is at most 1500 mOsm/L, or 1000 mOsm/L, or 900 mOsm/L

### Embodiment 25

The formulation according to any one of Embodiments 21-24, further comprising a sodium salt.

### Embodiment 26

The formulation according to Embodiment 25, wherein the sodium salt is sodium chloride or sodium acetate, or a mixture thereof.

### Embodiment 27

The formulation according to any one of Embodiments 21-26, where the polyol is a saccharide or a sugar alcohol.

### Embodiment 28

The formulation according to Embodiment 27, wherein the formulation contains sucrose in a concentration of at least 200 mM and at most 800 mM.

### Embodiment 29

The formulation according to Embodiment 27, wherein the formulation contains sorbitol in a concentration of at least 400 mM.

### Embodiment 30

The formulation according to any one of Embodiments 21-29, wherein the Factor VIII polypeptide is recombinant full length FVIII or a recombinant B-domain truncated FVIII.

### Embodiment 31

The formulation according to any one of Embodiments 21-30, wherein the Factor VIII polypeptide is attached to a protraction group.

### Embodiment 32

The formulation according to Embodiment 31, wherein the Factor VIII polypeptide is a pegylated FVIII, or an albumin-fused FVIII, or an Fc region-fused FVIII.

### Embodiment 33

The formulation according to Embodiment 31 or Embodiment 32, wherein the Factor VIII polypeptide is a glycopegylated B-domain truncated FVIII.

### Embodiment 34

The formulation according to any one of Embodiment 21-33, having a pH from 6.0 to 7.0, or from 6.4 to 7.0.

### Embodiment 35

A method for optimising a liquid formulation of coagulation Factor VIII, the method comprising the steps of:

(i) Providing a variety of liquid formulations comprising Factor VIII to be tested;
(ii) Adding a protein denaturant to said liquid formulations, and incubating the resulting solutions for a predetermined period of time;
(iii) Analysing the incubated solutions of (ii) for the presence of free FVIII light chain;
(iv) Selecting one or more formulation(s) having a desired low level of free light chain.

### Embodiment 36

A method for identifying a stable liquid formulation of Factor VIII, the method comprising the steps of:

(i) Providing a variety of liquid formulations comprising Factor VIII to be tested;
(ii) Adding a protein denaturant to said liquid formulations, and incubating the resulting solutions for a predetermined period of time;
(iii) Analysing the incubated solutions of (ii) for the presence of free FVIII light chain;
(iv) Selecting one or more formulation(s) having a desired low level of free light chain.

### Embodiment 37

The method according to Embodiment 35 or Embodiment 36, wherein the protein denaturant is guanidinium chloride or urea.

### Embodiment 38

The method according to any one of Embodiments 35-37, wherein the Factor VIII polypeptide is recombinant full length FVIII or a recombinant B-domain truncated FVIII.

### Embodiment 39

The method according to Embodiment 38, wherein the Factor VIII polypeptide is attached to a protraction group.

### Embodiment 40

The method according to Embodiment 39, wherein the Factor VIII polypeptide is a pegylated FVIII, or an albumin-fused FVIII, or an Fc region-fused FVIII.

### Embodiment 41

The method according to Embodiment 39 or Embodiment 40, wherein the Factor VIII polypeptide is a glycopegylated B-domain truncated FVIII.

### EXPERIMENTALS

### List of Abbreviations

SEC size-exclusion chromatography
LC light chain
GuHCl guanidinium chloride
BDD-FVIII B-domain deleted/truncated Factor VIII
GP-BDD-FVIII Glycopegylated B-domain truncated/deleted Factor VIII

### Example 1

### Production of Recombinant B-Domain Truncated/Deleted FVIII

B-domain truncated/deleted FVIII (“BDD-FVIII”) (SEQ ID NO 2):

Manufacture of BDD-FVIII is described e.g. by Thim L. et al. (Haemophilia 2010; 16:349-359)

Glycopegylated B-domain truncated/deleted FVIII (“GP-BDD-FVIII”):

Manufacture of GP-BDD-FVIII is described e.g. in International Publication WO 2009/108806.

FVIII-Fc/albumin fusion proteins:

Fusion proteins wherein Factor VIII is fused to an Fc domain (SEQ ID NO 4) or to albumin (SEQ ID NO 5), respectively, were prepared by transient expression in HEK cells followed by a three-step purification on an affinity column, F25 sepharose and Poros 50 HQ.

SEQI ID NO 4 - Fc fusion:
ATRRYYLGAVELSWDYMQSDLGELPVDARFPPRVPKSFPFNTSVVYKKTL
FVEFTDHLFNIAKPRPPWMGLLGPTIQAEVYDTVVITLKNMASHPVSLHA
VGVSYWKASEGAEYDDQTSQREKEDDKVFPGGSHTYVWQVLKENGPMASD
PLCLTYSYLSHVDLVKDLNSGLIGALLVCREGSLAKEKTQTLHKFILLFA
VFDEGKSWHSETKNSLMQDRDAASARAWPKMHTVNGYVNRSLPGLIGCHR
KSVYWHVIGMGTTPEVHSIFLEGHTFLVRNHRQASLEISPITFLTAQTLL
MDLGQFLLFCHISSHQHDGMEAYVKVDSCPEEPQLRMKNNEEAEDYDDDL
TDSEMDVVRFDDDNSPSFIQIRSVAKKHPKTWVHYIAAEEEDWDYAPLVL
APDDRSYKSQYLNNGPQRIGRKYKKVRFMAYTDETFKTREAIQHESGILG
PLLYGEVGDTLLIIFKNQASRPYNIYPHGITDVRPLYSRRLPKGVKHLKD
FPILPGEIFKYKWTVTVEDGPTKSDPRCLTRYYSSFVNMERDLASGLIGP
LLICYKESVDQRGNQIMSDKRNVILFSVFDENRSWYLTENIQRFLPNPAG
VQLEDPEFQASNIMHSINGYVFDSLQLSVCLHEVAYWYILSIGAQTDFLS
VFFSGYTFKHKMVYEDTLTLFPFSGETVFMSMENPGLWILGCHNSDFRNR
GMTALLKVSSCDKNTGDYYEDSYEDISAYLLSKNNAIEPRSFSQNSRHPS
QNPPVLKRHQR-
EITRTTLQSDQEEIDYDDTISVEMKKEDFDIYDEDENQSPRSFQKKTRHY
FIAAVERLWDYGMSSSPHVLRNRAQSGSVPQFKKVVFQEFTDGSFTQPLY
RGELNEHLGLLGPYIRAEVEDNIMVTFRNQASRPYSFYSSLISYEEDQRQ
GAEPRKNFVKPNETKTYFWKVQHHMAPTKDEFDCKAWAYFSDVDLEKDVH
SGLIGPLLVCHTNTLNPANGRQVTVQEFALFFTIFDETKSWYFTENMERN
CRAPCNIQMEDPTFKENYRFHAINGYIMDTLPGLVMAQDQRIRWYLLSMG
SNENIHSIHFSGHVFTVRKKEEYKMALYNLYPGVFETVEMLPSKAGIWRV
ECLIGEHLHAGMSTLFLVYSNKCQTPLGMASGHIRDFQITASGQYGQWAP
KLARLHYSGSINAWSTKEPFSWIKVDLLAPMIIHGIKTQGARQKFSSLYI
SQFIIMYSLDGKKWQTYRGNSTGTLMVFFGNVDSSGIKHNIFNPPIIARY
IRLHPTHYSIRSTLRMELMGCDLNSCSMPLGMESKAISDAQITASSYFTN
MFATWSPSKARLHLQGRSNAWRPQVNNPKEWLQVDFQKTMKVTGVTTQGV
KSLLTSMYVKEFLISSSQDGHQWTLFFQNGKVKVFQGNQDSFTPVVNSLD
PPLLTRYLRIHPQSVVVHQIALRMEVLGCEAQDLYGGGSGGGSGGGSGGG
SGGGSGGGSEPRGPTIKPCPPCKCPAPNAEGEPSVFIFPPKIKDVLMISL
SPMVTCVVVDVSEDDPDVQISWFVNNVEVLTAQTQTHREDYNSTLRVVSA
LPIQHQDWMSGKEFKCKVNNKALPAPIERTISKPKGSVRAPQVYVLPPPE
EEMTKKQVTLTCMVTDFMPEDIYVEWTNNGKTELNYKNTEPVLDSDGSYF
MYSKLRVEKKNWVERNSYSCSVVHEGLHNHHTTKSFSRTPGK
SEQ ID NO 5 - Albumin fusion:
ATRRYYLGAVELSWDYMQSDLGELPVDARFPPRVPKSFPFNTSVVYKKTL
FVEFTDHLFNIAKPRPPWMGLLGPTIQAEVYDTVVITLKNMASHPVSLHA
VGVSYWKASEGAEYDDQTSQREKEDDKVFPGGSHTYVWQVLKENGPMASD
PLCLTYSYLSHVDLVKDLNSGLIGALLVCREGSLAKEKTQTLHKFILLFA
VFDEGKSWHSETKNSLMQDRDAASARAWPKMHTVNGYVNRSLPGLIGCHR
KSVYWHVIGMGTTPEVHSIFLEGHTFLVRNHRQASLEISPITFLTAQTLL
MDLGQFLLFCHISSHQHDGMEAYVKVDSCPEEPQLRMKNNEEAEDYDDDL
TDSEMDVVRFDDDNSPSFIQIRSVAKKHPKTWVHYIAAEEEDWDYAPLVL
APDDRSYKSQYLNNGPQRIGRKYKKVRFMAYTDETFKTREAIQHESGILG
PLLYGEVGDTLLIIFKNQASRPYNIYPHGITDVRPLYSRRLPKGVKHLKD
FPILPGEIFKYKWTVTVEDGPTKSDPRCLTRYYSSFVNMERDLASGLIGP
LLICYKESVDQRGNQIMSDKRNVILFSVFDENRSWYLTENIQRFLPNPAG
VQLEDPEFQASNIMHSINGYVFDSLQLSVCLHEVAYWYILSIGAQTDFLS
VFFSGYTFKHKMVYEDTLTLFPFSGETVFMSMENPGLWILGCHNSDFRNR
GMTALLKVSSCDKNTGDYYEDSYEDISAYLLSKNNAIEPRSFSQNSRHPS
QNPPVLKRHQR-
EITRTTLQSDQEEIDYDDTISVEMKKEDFDIYDEDENQSPRSFQKKTRHY
FIAAVERLWDYGMSSSPHVLRNRAQSGSVPQFKKVVFQEFTDGSFTQPLY
RGELNEHLGLLGPYIRAEVEDNIMVTFRNQASRPYSFYSSLISYEEDQRQ
GAEPRKNFVKPNETKTYFWKVQHHMAPTKDEFDCKAWAYFSDVDLEKDVH
SGLIGPLLVCHTNTLNPAHGRQVTVQEFALFFTIFDETKSWYFTENMERN
CRAPCNIQMEDPTFKENYRFHAINGYIMDTLPGLVMAQDQRIRWYLLSMG
SNENIHSIHFSGHVFTVRKKEEYKMALYNLYPGVFETVEMLPSKAGIWRV
ECLIGEHLHAGMSTLFLVYSNKCQTPLGMASGHIRDFQITASGQYGQWAP
KLARLHYSGSINAWSTKEPFSWIKVDLLAPMIIHGIKTQGARQKFSSLYI
SQFIIMYSLDGKKWQTYRGNSTGTLMVFFGNVDSSGIKHNIFNPPIIARY
IRLHPTHYSIRSTLRMELMGCDLNSCSMPLGMESKAISDAQITASSYFTN
MFATWSPSKARLHLQGRSNAWRPQVNNPKEWLQVDFQKTMKVTGVTTQGV
KSLLTSMYVKEFLISSSQDGHQWTLFFQNGKVKVFQGNQDSFTPVVNSLD
PPLLTRYLRIHPQSVVVHQIALRMEVLGCEAQDLYGGGSGGGSGGGSGGG
SGGGSGGGSDAHKSEVAHRFKDLGEENFKALVLIAFAQYLQQCPFEDHVK
LVNEVTEFAKTCVADESAENCDKSLHTLFGDKLCTVATLRETYGEMADCC
AKQEPERNECFLQHKDDNPNLPRLVRPEVDVMCTAFHDNEETFLKKYLYE
IARRHPYFYAPELLFFAKRYKAAFTECCQAADKAACLLPKLDELRDEGKA
SSAKQRLKCASLQKFGERAFKAWAVARLSQRFPKAEFAEVSKLVTDLTKV
HTECCHGDLLECADDRADLAKYICENQDSISSKLKECCEKPLLEKSHCIA
EVENDEMPADLPSLAADFVESKDVCKNYAEAKDVFLGMFLYEYARRHPDY
SVVLLLRLAKTYETTLEKCCAAADPHECYAKVFDEFKPLVEEPQNLIKQN
CELFEQLGEYKFQNALLVRYTKKVPQVSTPTLVEVSRNLGKVGSKCCKHP
EAKRMPCAEDYLSVVLNQLCVLHEKTPVSDRVTKCCTESLVNRRPCFSAL
EVDETYVPKEFNAETFTFHADICTLSEKERQIKKQTALVELVKHKPKATK
EQLKAVMDDFAAFVEKCCKADDKETCFAEEGKKLVAASQAALGL

### Example 2

### FVIIIa Activity Assay: Chromogenic Assay

The FVIII activity (FVIII:C) of the rFVIII compound is evaluated in a chromogenic FVIII assay using Coatest® SP FVIII reagents (Chromogenix) as follows: rFVIII samples and a FVIII standard (e.g. purified wild-type rFVIII calibrated against the 7th international FVIII standard from NIBSC) are diluted in Coatest® assay buffer (50 mM Tris, 150 mM NaCl, 1% BSA, pH 7.3, with preservative). Fifty μl of samples, standards, and buffer negative control are added to 96-well microtiter plates (Nunc) in duplicates. The factor IXa/factor X reagent, the phospholipid reagent and CaCl₂ from the Coatest® SP kit are mixed 5:1:3 (vol:vol:vol) and 75 μl of this added to the wells. After 15 min incubation at room temperature, 50 μl of the factor Xa substrate S-2765/thrombin inhibitor 1-2581 mix is added and the reagents incubated for 10 minutes at room temperature before 25 μl 1 M citric acid, pH 3, is added. The absorbance at 415 nm is measured on a Spectramax® microtiter plate reader (Molecular Devices) with absorbance at 620 nm used as reference wavelength. The value for the negative control is subtracted from all samples and a calibration curve prepared by linear regression of the absorbance values plotted vs. FVIII concentration.

### Example 3

### FVIIIa Activity Assay: One-Stage Clot Assay

FVIII activity (FVIII:C) of the rFVIII compounds is further evaluated in a one-stage FVIII clot assay as follows: rFVIII samples and a FVIII standard (e.g. purified wild-type rFVIII calibrated against the 7th international FVIII standard from NIBSC) are diluted in HBS/BSA buffer (20 mM hepes, 150 mM NaCl, pH 7.4 with 1% BSA) to approximately 10 U/ml, followed by 10-fold dilution in FVIII-deficient plasma containing VWF (Dade Behring). Samples are subsequently diluted in HBS/BSA buffer. The APTT clot time is measured using an ACL300R or an ACL5000 instrument (Instrumentation Laboratory) using the single factor programme. FVIII-deficient plasma with VWF (Dade Behring) is used as assay plasma and SynthASil®, (Hemosil®, Instrumentation Laboratory) as a PTT reagent. In the clot instrument, the diluted sample or standard is mixed with FVIII-deficient plasma and a PTT reagent at 37° C. Calcium chloride is added and time until clot formation is determined by measuring turbidity. The FVIII:C in the sample is calculated based on a standard curve of the clot formation times of the dilutions of the FVIII standard.

### Example 4

### FVIII Degradation: Determination of FVIII Free Light Chain by Size-Exclusion Chromatography (SEC)

The dissociation of the rFVIII compound into free heavy and light chains is evaluated by a SEC method. The column is Sepax Zenix™ SEC-300 and the eluent is 10 mM Tris, 10 mM CaCl₂, 300 mM NaCl and 5% isopropanol, pH 7.0 Degradation of Factor VIII molecules is observed in SEC as appearance of a peak with longer elution times than monomeric Factor VIII. This peak has been assigned to free Light Chain (free LC).

### Working Examples

### Example 5

A series of formulations of glycopegylated Factor VIII (GP-BDD-FVIII) were prepared with the following components in all formulations: 28 μg/mL glycopegylated Factor VIII, 18 mg/mL NaCl, 0.1 mg/mL polysorbate 80, 0.6 mg/mL sucrose, 0.055 mg/mL Methionine, 1.5 mg/mL Histidine, 0.13 mg/mL CaCl₂, pH 6.5. In addition, each formulation contained an additional polyol stabilizer as listed in the following table 1:

TABLE 1

| | |
|---|---|
| Formulation 1 | No additional stabilizer |
| Formulation 2 | 0.3M sucrose |
| Formulation 3 | 1.0M sucrose |
| Formulation 4 | 1.0M glycerol |
| Formulation 5 | 0.3M raffinose |
| Formulation 6 | 0.3M stachyose |

The samples were incubated for 5 weeks at 5° C. and analysed for free Light Chain by SEC (above). In addition, a set of samples with identical formulations, but also containing 0.2 M GuHCl, were prepared and incubated 24 h at 5° C., and then analysed for free Light Chain by SEC. The relative area of the free Light Chain peak in the two experiments is listed in the following table 2:

TABLE 2

| Formulation | 5 weeks at 5° C. | 24 h at 5° C. |
|---|---|---|
| 1 | 5.40% | 2.16% |
| 2 | 3.27% | 1.36% |
| 3 | 0.51% | 0.19% |
| 4 | 2.90% | 1.50% |
| 5 | 2.02% | 1.41% |
| 6 | 1.30% | 1.00% |

It can be seen that formulation 3 with the lowest formation of free Light Chain during 5 weeks at 5° C. is correctly identified by the rapid method with chemical denaturant.

### Example 6

In order to investigate the optimal Calcium concentration in a liquid formulation of glycopegylated Factor VIII (GP-BDD-FVIII), formulations were prepared with about 250 U/mL glycopegylated Factor VIII, 18 mg/mL NaCl, 0.05 mg/mL polysorbate 80, 1.5 mg/mL sucrose, 1 mg/mL Methionine, and 1.5 mg/mL Histidine pH 6.9. The Calcium Chloride concentrations are listed in the following table 3:

TABLE 3

| | |
|---|---|
| Formulation 1 | 3 mM CaCl₂ |
| Formulation 2 | 10 mM CaCl₂ |
| Formulation 3 | 30 mM CaCl₂ |
| Formulation 4 | 100 mM CaCl₂ |

The solutions were sterile filtered, distributed in vials, and degassed by 3 cycles of exposure to pure N₂, interspersed by brief evacuation of the chamber to 0.1 bar pressure, and the vials were sealed with pure N₂ in the headspace. The samples were analysed by SEC HPLC (above) after 8 weeks at 30° C. and 26 weeks at 5° and for activity (chromogenic assay, above) after 37 weeks at 5° C. Furthermore, a rapid screening experiment was set up with the same Calcium concentrations and 130 μg/mL glycopegylated Factor VIII, 0.2 M GuHCl, 18 mg/mL NaCl, 0.1 mg/mL polysorbate 80, 3 mg/mL sucrose, 0.055 mg/mL Methionine, and 1.5 mg/mL Histidine, pH 6.9. Samples were incubated for 24 h at 5° C.

The table 4 below lists the relative amounts of free Light Chain and Factor VIII activity obtained under different conditions.

TABLE 4

| CalCl2 | Activity, | Free Light | Free Light | Free Light |
|---|---|---|---|---|
| concen- | 37 weeks | Chain, 26 weeks | Chain, 8 weeks | Chain, 0.2M |
| tration | at 5° C. | at 5° C. | at 30° C. | GuHCl |
| 3 mM | 128 IU/ml | 11.9% | 4.5% | 2.3% |
| 10 mM | 189 IU/ml | 6.8% | 4.4% | 1.5% |
| 30 mM | 198 IU/ml | 4.8% | 5.1% | 0.7% |
| 100 mM | 177 IU/ml | 5.4% | 11.1% | 0.7% |

At 5° C., increasing the Calcium concentration from 3 to 10 mM clearly gives better conservation of activity over 37 weeks and lower formation of free Light Chain over 26 weeks. This can be predicted after only 24 h by the assay with chemical denaturant, while accelerated stability at 30° C. only shows a marginal difference. A further stabilization is obtained by going from 10 to 30 mM Calcium. Again, this is well predicted by the chemical denaturation assay, but not by accelerated stability at 30° C.

### Example 7

The stabilizing effect of saccharides was investigated by preparing formulations with 130 μg/mL glycopegylated Factor VIII (GP-BDD-FVIII), 0.4 M GuHCl, 18 mg/mL NaCl, 3.9 mM CaCl₂, 0.1 mg/mL polysorbate 80, 3 mg/mL sucrose, 0.055 mg/mL Methionine, and 1.5 mg/mL Histidine, pH 6.9 and different additional concentrations of saccharides. Samples were incubated for 24 h at 5° C. and analyzed by SEC (above). The relative areas of free Light Chain obtained with the different stabilizers are listed in the following table 5:

TABLE 5

| | Free Light Chain, |
|---|---|
| Stabilizer | 24 h at 5° C., 0.4M GuHCl |
| No stabilizer | 5.1% |
| 0.2M sucrose | 2.6% |
| 0.4M sucrose | 1.9% |
| 0.6M sucrose | 0.9% |
| 0.8M sucrose | 0.7% |
| 0.15M raffinose | 3.2% |
| 0.3M raffinose | 1.9% |
| 0.45M raffinose | 1.2% |
| 0.1M stachyose | 4.2% |
| 0.2M stachyose | 2.5% |
| 0.3M stachyose | 2.0% |

It is seen that all three saccharides are efficient in stabilizing liquid formulation of Factor VIII.

### Example 8

The effect of pH, NaCl concentration, Sodium acetate (NaOAc) concentration, Calcium Chloride concentration and sucrose concentration on liquid stability of glycopegylated Factor VIII (GP-BDD-FVIII) was investigated in the presence of 0.35 M GuHCl in a multifactorial experiment.

All samples contained 150 μg/mL GP-BDD-FVIII, and 0.1 mg/mL polysorbate 80. Other components are given in the table 6 below. All these formulations have calculated osmotic concentration below 900 mOsm/L, without taking into account the content of GuHCl, which is not part of the pharmaceutical formulation being developed. The samples were incubated for 5 days at 5° C. and analyzed by SEC (as described above). The relative area of the free Light Chain peak is also listed in the table.

TABLE 6

| | Histidine | NaCl | NaOAc | Calcium | Sucrose | | Free |
|---|---|---|---|---|---|---|---|
| Formulation | (mM) | (mM) | (mM) | (mM) | (mM) | pH | LC |
| 1 | 10 | 200 | — | 10 | 0 | 6.4 | 5.2% |
| 2 | 10 | 200 | — | 10 | 300 | 6.4 | 3.3% |
| 3 | 10 | 200 | — | 10 | 600 | 6.4 | 1.7% |
| 4 | 10 | 200 | — | 10 | 0 | 6.7 | 5.7% |
| 5 | 10 | 200 | — | 10 | 300 | 6.7 | 3.9% |
| 6 | 10 | 200 | — | 10 | 600 | 6.7 | 1.9% |
| 7 | 10 | 200 | — | 10 | 0 | 7.0 | 7.9% |
| 8 | 10 | 200 | — | 10 | 300 | 7.0 | 4.1% |
| 9 | 10 | 200 | — | 10 | 600 | 7.0 | 2.2% |
| 10 | 10 | 200 | — | 30 | 0 | 6.4 | 3.2% |
| 11 | 10 | 200 | — | 30 | 300 | 6.4 | 2.1% |
| 12 | 10 | 200 | — | 30 | 600 | 6.4 | 1.1% |
| 13 | 10 | 200 | — | 30 | 0 | 6.7 | 3.0% |
| 14 | 10 | 200 | — | 30 | 300 | 6.7 | 1.7% |
| 15 | 10 | 200 | — | 30 | 600 | 67 | 1.3% |
| 16 | 10 | 200 | — | 30 | 0 | 7.0 | 3.6% |
| 17 | 10 | 200 | — | 30 | 300 | 7.0 | 2.3% |
| 18 | 10 | 200 | — | 30 | 600 | 7.0 | 1.4% |
| 19 | — | — | 200 | 10 | 0 | 6.7 | 4.9% |
| 20 | — | — | 200 | 10 | 300 | 6.7 | 2.8% |
| 21 | — | — | 200 | 10 | 600 | 6.7 | 1.4% |
| 22 | — | — | 200 | 10 | 0 | 7.0 | 4.6% |
| 23 | — | — | 200 | 10 | 300 | 7.0 | 2.5% |
| 24 | — | — | 200 | 10 | 600 | 7.0 | 1.4% |
| 25 | — | — | 200 | 10 | 0 | 7.2 | 5.5% |
| 26 | — | — | 200 | 10 | 300 | 7.2 | 3.0% |
| 27 | — | — | 200 | 10 | 600 | 7.2 | 1.5% |
| 28 | — | — | 200 | 30 | 0 | 6.7 | 2.9% |
| 29 | — | — | 200 | 30 | 300 | 6.7 | 1.5% |
| 30 | — | — | 200 | 30 | 600 | 6.7 | 1.0% |
| 31 | — | — | 200 | 30 | 0 | 7.0 | 2.3% |
| 32 | — | — | 200 | 30 | 300 | 7.0 | 1.4% |
| 33 | — | — | 200 | 30 | 600 | 7.0 | 1.1% |
| 34 | — | — | 200 | 30 | 0 | 7.2 | 2.5% |
| 35 | — | — | 200 | 30 | 300 | 7.2 | 1.8% |
| 36 | — | — | 200 | 30 | 600 | 7.2 | 1.2% |
| 37 | 10 | — | — | 10 | 0 | 6.4 | 9.6% |
| 38 | 10 | — | — | 10 | 300 | 6.4 | 6.6% |
| 39 | 10 | — | — | 10 | 600 | 6.4 | 3.2% |
| 40 | 10 | — | — | 10 | 0 | 6.7 | 9.8% |
| 41 | 10 | — | — | 10 | 300 | 6.7 | 5.8% |
| 42 | 10 | — | — | 10 | 600 | 6.7 | 3.0% |
| 43 | 10 | — | — | 10 | 0 | 7.0 | 8.6% |
| 44 | 10 | — | — | 10 | 300 | 7.0 | 5.0% |
| 45 | 10 | — | — | 10 | 600 | 7.0 | 3.6% |
| 46 | 10 | — | — | 30 | 0 | 6.4 | 7.7% |
| 47 | 10 | — | — | 30 | 300 | 6.4 | 3.6% |
| 48 | 10 | — | — | 30 | 600 | 6.4 | 2.3% |
| 49 | 10 | — | — | 30 | 0 | 6.7 | 4.4% |
| 50 | 10 | — | — | 30 | 300 | 6.7 | 3.3% |
| 51 | 10 | — | — | 30 | 600 | 6.7 | 1.9% |
| 52 | 10 | — | — | 30 | 0 | 7.0 | 4.4% |
| 53 | 10 | — | — | 30 | 300 | 7.0 | 3.3% |

It can be seen that the highest formation of free Light Chain is observed in the absence of NaCl and NaOAc, and with 10 mM CaCl₂ and no sucrose (formulation 37, 40 and 43). Increasing Calcium concentration to 30 mM, adding sucrose to 300 or 600 mM and adding NaCl or NaOAc all decreases formation of free Light Chain. The slowest formation of free Light Chain is observed with 200 mM NaOAc, 30 mM CaCl₂ and 600 mM sucrose (formulations 30, 33, and 36).

Almost as good are samples with 200 mM NaCl, 30 mM CaCl₂ and 600 mM sucrose (formulations 12, 15 and 18). The differences between the different values of pH are within the variation of the experiment. These results confirm the stabilizing effect of 30 mM Calcium and 300 or preferably 600 mM sucrose, and also suggest that the complete absence of Sodium is detrimental to the stability of Factor VIII in aqueous solution.

### Example 9

In order to assess the optimal concentration of NaCl or NaOAc, an experiment with urea as chemical denaturant was performed. Since GuHCl is itself a salt, it might interfere with determination of the optimal concentration of other salts.

All samples contained 100 μg/mL glycopegylated FVIII (GP-BDD-FVIII), 1.6 M urea, 30 mM CaCl₂, 570 mM sucrose, and 0.1 mg/mL polysorbate 80. The concentrations of Histidine, NaCl and Na acetate (NaOAc) are listed in the table 7 below, together with the relative area of free Light Chain after 96 hours at 5° C. The content of free LC was measured by SEC (above). The table also lists the osmostic concentration calculated as described above, without taking into account the content of urea, which is not part of the pharmaceutical formulation being developed.

TABLE 7

| | | | | | Calculated | Free |
|---|---|---|---|---|---|---|
| | Histidine | NaCl | NaOAc | | osmotic | Light |
| Formulation | (mM) | (mM) | (mM) | pH | concentration | Chain |
| 1 | 10 | 0 | — | 6.7 | 669 mOsm/L | 2.7% |
| 2 | 10 | 10 | — | 6.7 | 687 mOsm/L | 1.9% |
| 3 | 10 | 20 | — | 6.7 | 706 mOsm/L | 1.8% |
| 4 | 10 | 40 | — | 6.7 | 743 mOsm/L | 1.9% |
| 5 | 10 | 80 | — | 6.7 | 818 mOsm/L | 2.0% |
| 6 | 10 | 160 | — | 6.7 | 966 mOsm/L | 2.1% |
| 7 | — | — | 10 | 6.8 | 678 mOsm/L | 1.8% |
| 8 | — | — | 20 | 6.8 | 697 mOsm/L | 1.9% |
| 9 | — | — | 40 | 6.9 | 735 mOsm/L | 1.8% |
| 10 | — | — | 80 | 6.9 | 811 mOsm/L | 1.7% |
| 11 | — | — | 160 | 7.0 | 963 mOsm/L | 1.6% |

It can be seen that formation of free Light Chain is highest in the formulation without any Sodium salt. The addition of 10 mM NaCl or 10 mM NaOAc improves the stability. Further addition of NaCl up to 160 mM does not stabilize further and possibly destabilizes slightly, while addition of NaOAc up to 160 mM stabilizes slightly.

### Example 10

A number of formulations of GP-BDD-FVIII were prepared. All formulations contained 10 mM L-histidine, 0.02 mg/mL polysorbate 80, 0.5 mg/mL poloxamer 188, 0.37 mM L-methionine, 310 mM NaCl, and 0.6 mg/mL sucrose, and had pH adjusted to 6.4. The formulations contained about 250 U/mL of GP-BDD-FVIII, and different concentrations of CaCl₂. The solutions were sterile filtered, distributed in vials, and degassed by 3 cycles of exposure to pure N₂, interspersed by brief evacuation of the chamber to 0.1 bar pressure. The vials were closed with N₂ in the headspace and incubated at 5° C. After 4 and 8 weeks of incubation, the samples were analysed by size-exclusion chromatography (SEC, above). Table 8 below shows the relative area of this peak for the different formulations:

TABLE 8

| | | | | | | | |
|---|---|---|---|---|---|---|---|
| Form- | 1 | 2 | 3 | 4 | 5 | 6 | 7 |
| ulation | 10 | 15 | 20 | 30 | 45 | 60 | 100 |
| [CaCl₂] | mM | mM | mM | mM | mM | mM | mM |

% Free Light Chain

| | | | | | | | |
|---|---|---|---|---|---|---|---|
| Time zero | 1.0 | 0.95 | 0.93 | 0.88 | 0.82 | 0.71 | 0.99 |
| 4 weeks | 3.19 | 2.49 | 2.46 | 2.52 | 2.28 | 2.6 | 2.61 |
| 8 weeks | 3.55 | 3.05 | 2.7 | 2.38 | 2.43 | 2.28 | 2.39 |

It is seen that the amount of free light chain increases slower at 15 mM CaCl₂ than at 10 mM CaCl₂, slower yet at 20 mM CaCl₂ and even slower at 30 mM CaCl₂. The variation in free light chain formation between 30, 45, 60 and 100 mM CaCl₂ is within the experimental uncertainty.

### Example 11

A series of formulations of Factor VIII fused to an Fc domain (SEQ ID NO 4) or to albumin (SEQ ID NO 5) were prepared. These proteins have putative long duration of action. Formulations of the Fc fusion protein contained about 200 U/ml Factor VIII derivative, 10 mM imidazole, pH 7.3, 0.1 mg/ml polysorbate 80, 0.5 M glycerol, 0.25 M NaCl and 0.6 M GuHCl. Formulations of the albumin fusion protein contained about 500 U/ml Factor VIII derivative, 10 mM imidazole, pH 7.3, 0.1 mg/ml polysorbate 80, 0.5 M glycerol, 0.25 M NaCl and 0.6 M GuHCl. In addition, the formulations contained different concentrations of sucrose and CaCl₂. The formulations were incubated for about 24 h at 5° C. and analysed by size-exclusion chromatography (SEC, above). The following Table 9 lists the CaCl₂ and sucrose concentration as well as the relative light chain area measured

TABLE 9

| | CaCl₂ | Sucrose | |
|---|---|---|---|
| Factor VIII | concentration | concentration | % Free |
| derivative | (mM) | (mM) | light chain |
| FVIII-Fc | 5 | 0 | 38.7% |
| FVIII-Fc | 5 | 500 | 21.5% |
| FVIII-Fc | 30 | 0 | 17.4% |
| FVIII-Fc | 30 | 500 | 7.5% |
| FVIII-albumin | 5 | 0 | 50.1% |
| FVIII-albumin | 5 | 500 | 29.2% |
| FVIII-albumin | 30 | 0 | 25.0% |
| FVIII-albumin | 30 | 500 | 10.0% |

It is seen that the accelerated assay also is applicable to these Factor VIII derivatives. It is also seen that Calcium concentrations of 30 mM and sucrose concentrations of 500 mM have a beneficial effect on the liquid stability of the Factor VIII derivatives.

### Example 12

A series of formulations of BDD-FVIII were prepared. All formulations shared the following components: About 2500 IU/ml BDD-FVIII, 310 mM NaCl, 20 mM Histidine, 6 mg/ml sucrose, 0.11 mg/ml Methionine, 0.2 mg/ml polysorbate 80 and 0.4 M Guanidine hydrochloride. The formulations furthermore contained different concentrations of calcium chloride in the range 3-100 mM. The formulations were incubated at 5° C. for about 24 hours and analysed by size-exclusion chromatography (SEC, above). The following Table 10 lists the relative integral of the light chain for different concentrations of CaCl₂:

TABLE 10

| CaCl₂ concentration (mM) | % Free light chain |
|---|---|
| 3 | 7.8% |
| 5 | 5.4% |
| 10 | 3.8% |
| 20 | 3.0% |
| 30 | 2.8% |
| 50 | 2.5% |
| 100 | 2.6% |

It is seen that BDD-FVIII is stabilized by increasing the concentration of Calcium above 10 mM, with values 30-100 mM being about equally effective.

### Example 13

A series of formulations of BDD-FVIII were prepared. All formulations shared the following components: About 500 IU/ml BDD-FVIII, 310 mM NaCl, 7 mM Histidine, 2 mg/ml sucrose, 0.04 mg/ml Methionine, 0.07 mg/ml polysorbate 80 and 0.6 M Guanidine hydrochloride. The formulations furthermore contained different concentrations of calcium chloride in the range 1-30 mM. The samples were incubated for 4 days at 5° C. and assayed for Factor VIII activity by chromogenic assaying (Coatest® SP FVIII, above). The measured activities are listed in the following table:

TABLE 11

| CaCl₂ concentration (mM) | Activity (U/ml) |
|---|---|
| 1 | 3 |
| 3 | 455 |
| 10 | 442 |
| 30 | 522 |

It can be seen that the accelerated stability screening can also be performed using a biological activity assay.

### Example 14

A series of formulations of BDD-FVIII were prepared. All formulations shared the following components: About 2000 IU/ml BDD-FVIII, 0.6 M GuHCl, 30 mM CaCl₂, 570 mM sucrose, 0.1 mg/ml polysorbate 80, 40 mM NaCl and 10 mM Histidine. The value of pH varied between 5.5 and 7.2. All formulations had a calculated osmotic concentration of about 743 mOsm/L, without taking into account the content of GuHCl, which is not part of the pharmaceutical formulation being developed. The formulations were incubated at 5° C. for 3 days and analysed by size-exclusion chromatography (SEC, above). The following table lists the relative integral of the light chain for different pH values:

TABLE 12

| pH | Relative area of Light Chain |
|---|---|
| 5.52 | 10.19% |
| 5.81 | 6.82% |
| 6.09 | 5.95% |
| 6.43 | 5.00% |
| 6.66 | 4.87% |
| 6.76 | 5.32% |
| 6.94 | 6.84% |
| 7.18 | 8.49% |
| 7.20 | 9.16% |

It can be seen that pH values around 6.5 are most favourable.

### Example 15

Two formulations of GP-BDD-FVIII were prepared. Both formulations contained about 290 U/ml GP-BDD_FVIII, 10 mM Histidine, 30 mM CaCl₂, 1.5 mg/ml pluronic F68, 600 mM sucrose and had pH adjusted to 6.7. One formulation was without NaCl, the other with 78 mM NaCl. The formulation without NaCl had a calculated osmotic concentration of about 700 mOsm/L, the formulation with 78 mM NaCl had a calculated osmotic concentration of about 845 mOsm/L. The solutions were sterile filtered, distributed in vials, and degassed by 3 cycles of exposure to pure N₂, interspersed by brief evacuation of the chamber to 0.1 bar pressure, and the vials were sealed with pure N₂ in the headspace. Samples were incubated at 5° C. or −80° C. for 52 weeks and the activity was measured with the chromogenic assay. The table below lists the results.

TABLE 13

| | 24 | 24 | 52 | 52 |
|---|---|---|---|---|
| | weeks, −80° | weeks, 5° | weeks, −80° | weeks, 5° |
| | C. | C. | C. | C. |
| 78 mM NaCl | 296 U/ml | 292 U/ml | 315 U/ml | 297 U/ml |
| No NaCl | 287 U/ml | 276 U/ml | 304 U/ml | 264 U/ml |

It can be seen that the activity is preserved very well in both formulations, with the samples stored at 5° C. showing essentially the same activity as the reference stored at −80° C.

### Example 16

Eight formulations of GP-BDD-FVIII were prepared. All formulations contained about 300 U/ml GP-BDD-FVIII, 30 mM CaCl₂, 0.1 mg/ml polysorbate 80 and 500 mM sucrose. The formulations contained different concentrations of Histidine, NaCl, Na acetate, and were adjusted to different values of pH, as detailed in the table below. The solutions were sterile filtered, distributed in vials, and degassed by 3 cycles of exposure to pure Nₐ interspersed by brief evacuation of the chamber to 0.1 bar pressure, and the vials were sealed with pure N₂ in the headspace. Samples were incubated at 5° or −80° C. for 32 weeks and the activity was measured with the chromogenic method. The results are also listed in the table

TABLE 14

| | | | | | | | | |
|---|---|---|---|---|---|---|---|---|
| Histidine (mM) | 10 | 10 | 10 | 10 | — | — | — | — |
| NaCl (mM) | 150 | 150 | 150 | 150 | — | — | — | — |
| Na acetate (mM) | — | — | — | — | 155 | 155 | 155 | 155 |
| pH | 6.2 | 6.5 | 6.7 | 7.2 | 6.4 | 6.9 | 7.2 | 7.3 |
| Calculated osmostic coefficient | 876 | 876 | 876 | 876 | 882 | 882 | 882 | 882 |
| (mOsm/L) | | | | | | | | |
| Activity, 32 weeks, −80° C. (U/ml) | 283 | 287 | 315 | 333 | 312 | 302 | 345 | 313 |
| Activity, 32 weeks, 5° C. (U/ml) | 265 | 294 | 283 | 261 | 289 | 294 | 295 | 258 |

It can be seen that the samples stored at 5° C. show almost the same activity as the reference stored at −80° C., with the activity being particularly well preserved at pH 6.4-6.9.

### Example 17

Eight formulations of GP-BDD-FVIII were prepared. All formulations contained about 290 U/ml GP-BDD_FVIII, 0.3 mM Methionine, 30 mM CaCl₂, 0.1 mg/ml polysorbate 80 and 10 mM Histidine and had pH of 6.5. The formulations contained different concentrations of sucrose and NaCl, as detailed in the table below. The solutions were sterile filtered, distributed in vials, and degassed by 3 cycles of exposure to pure N₂, interspersed by brief evacuation of the chamber to 0.1 bar pressure, and the vials were sealed with pure N₂ in the headspace. Samples were incubated at 5° or −80° C. for 32 weeks and the activity was measured with the chromogenic method. The results are also listed in the table

TABLE 15

| | | | | | | | |
|---|---|---|---|---|---|---|---|
| NaCl (mM) | 310 | 78 | 78 | 155 | 155 | 310 | 310 |
| Sucrose (mM) | 9 | 500 | 1000 | 500 | 1000 | 500 | 1000 |
| Calculated osmotic coefficient | 673 | 742 | 1252 | 885 | 1377 | 1174 | 1684 |
| (mOsm/L) | | | | | | | |
| Activity 32 weeks −80° C. (U/ml) | 281 | 287 | 287 | 293 | 283 | 295 | 289 |
| Activity 32 weeks 5° C. (U/ml) | 232 | 277 | 268 | 274 | 283 | 283 | 272 |

It can be seen that the samples stored at 5° C. show almost the same activity as the reference stored at −80° C., except for the formulation with 9 mM sucrose. Clearly, higher sucrose concentrations imparts high stability to the formulations, even at NaCl concentrations much lower than the values previously described for stable, liquid factor VIII formulations.

### Example 18

Four formulations of full-length Factor VIII (Kogenate™) were prepared. All formulations contained 500 IU/ml Factor VIII, 0.6 M GuHCl, 20 mM Histidine, 38 mM NaCl, 0.1 mg/ml polysorbate 80, pH 6.9. In addition, the formulations contained different amounts of CaCl₂ and sucrose. The formulations were incubated for about 24 h at 5°, and assayed for free LC content by SEC chromatography. The results are listed in the table below

TABLE 16

| Formulation | [CaCl₂] (mM) | [sucrose] (mM) | % Free LC |
|---|---|---|---|
| 1 | 3 | 15 | 25.4% |
| 2 | 3 | 500 | 12.4% |
| 3 | 28 | 15 | 13.8% |
| 4 | 28 | 500 | 8.5% |

It is seen that full-length Factor VIII also is stabilized against dissociation of light chain from heavy chain by increased Calcium and sucrose concentrations, and particularly stabilized when both Calcium and sucrose concentrations are increased.

### Example 19

Six formulations of GP-BDD-FVIII were prepared. All formulations contained 30 mM CaCl₂ and 0.055 mg/ml L-Methionine. Other components were as listed in the table below. The solutions were sterile filtered, distributed in vials, and degassed by 3 cycles of exposure to pure N₂, interspersed by brief evacuation of the chamber to 0.1 bar pressure, and the vials were sealed with pure N₂ in the headspace. The formulations were stored for 9 months at −80° C. or 5° C. and assayed for activity by the chromogenic assay. The results are listed in the table.

TABLE 17

| Formulation | 1 | 2 | 3 | 4 | 5 | 6 |
|---|---|---|---|---|---|---|
| GP-BDD-FVIII | 200 U/ml | 700 U/ml | 200 U/ml | 700 U/ml | 200 U/ml | 700 U/ml |
| Histidine (mM) | 10 | 10 | 10 | 10 | | |
| Sucrose (mM) | 570 | 570 | 660 | 660 | 570 | 570 |
| Polysorbate 80 | 0.1 | 0.1 | | | 0.1 | 0.1 |
| (mg/ml) | | | | | | |
| Poloxamer 188 | | | 0.5 | 0.5 | | |
| NaCl (mM) | 78 | 78 | 78 | 78 | | |
| Na acetate (mM) | | | | | 80 | 80 |
| pH | 6.7 | 6.7 | 6.7 | 6.7 | 6.4 | 6.4 |
| Calculated | 815 | 815 | 906 | 906 | 821 | 821 |
| osmotic | mOsm/L | mOsm/L | mOsm/L | mOsm/L | mOsm/L | mOsm/L |
| concentration | | | | | | |
| Activity, 9 months | 186 | 743 | 208 | 669 | 191 | 593 |
| −80° C. | | | | | | |
| Activity, 9 months | 169 | 610 | 209 | 710 | 174 | 514 |
| 5° C. | | | | | | |

It is seen that the activity is preserved well after 9 months at 5° C.

### Example 20

Two formulations of BDD-FVIII were prepared. Both formulations contained 30 mM CaCl₂, 10 mM Histidine, 570 mM sucrose, 78 mM NaCl, 0.1 m/ml polysorbate 80 and 0.055 mg/ml L-Methionine, and had pH adjusted to 6.7. The formulations had different concentrations of Factor VIII as listed in the table below. The solutions were sterile filtered, distributed in vials, and degassed by 3 cycles of exposure to pure N₂, interspersed by brief evacuation of the chamber to 0.1 bar pressure, and the vials were sealed with pure N₂ in the headspace. The formulations were stored for 9 months at −80° C. or 5° C. and assayed for activity by the chromogenic assay. The results are listed in the table.

TABLE 18

| Formulation | 1 | | 2 | |
|---|---|---|---|---|
| Factor VIII concentration | 250 | IU/ml | 700 | IU/ml |
| Calculated osmotic | 815 | mOsm/L | 815 | mOsm/L |
| concentration | | | | |
| Activity, 9 months −80° C. | 234 | IU/ml | 663 | IU/ml |
| Activity, 9 months 5° C. | 249 | IU/ml | 723 | IU/ml |

It is seen that the activity is preserved well after 9 months at 5° C.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A liquid, aqueous formulation of coagulation Factor VIII, comprising a Factor VIII molecule; a calcium salt in a concentration of more than 10 mM; and a saccharide and/or polyol in a concentration of at least 100 mM; wherein the formulation has a pH from 5.5-7.5.

2. A liquid, aqueous formulation of coagulation Factor VIII according to claim 1, comprising a Factor VIII molecule; a calcium salt in a concentration of at least 15 mM; and a saccharide and/or polyol in a concentration of at least 100 mM; wherein the formulation has a pH from 5.5-7.5.

3. The formulation of claim 1 or claim 2, wherein the calcium salt is present in a concentration of 15-100 mM, or 15-80 mM, or 15-60 mM, or 15-45 mM, or 20-100 mM, or 20-80 mM, or 20-60 mM, or 20-45 mM, or 20-40 mM, or 25-35 mM.

4. The formulation according to claim 1, wherein the calcium salt is calcium acetate, calcium lactate, calcium benzoate, calcium chloride, or a mixture of two or more thereof.

5. The formulation according to claim 4, wherein the salt is calcium chloride.

6. The formulation according to claim 1, further comprising a sodium salt in a concentration of at least 5 mM.

7. The formulation according to claim 1, wherein the sodium salt is present in a concentration of 5-500 mM, or 15-200 mM, or 15-150 mM, or 15-100 mM, or 50-150 mM, or 5-50 mM.

8. The formulation according to claim 6, wherein the sodium salt is sodium chloride, sodium acetate, or a mixture thereof.

9. The formulation according to claim 1, wherein the polyol is a mono- or disaccharide, a sugar alcohol, or a combination thereof.

10. The formulation according to claim 9, wherein the mono- or disaccharide and/or the sugar alcohol is selected from sucrose, sorbitol, glycerol, raffinose, stachyose, mannitol, sorbitol, or mixtures thereof.

11. The formulation according to claim 9, wherein the mono- or disaccharide and/or the sugar alcohol is present in a concentration of at least 100 mM, or at least 200 mM, or 100-1800 mM, or 300-1800 mM, or 100-1500 mM, or 200-1800 mM, or 200-1500 mM, or 100-1000 mM, or 200-1000 mM, or 300-1000 mM, or 200-800 mM, or 300-800 mM, or 400-800 mM, or 500-800 mM, or 500-700 mM.

12. The formulation according to claim 1, wherein the formulation contains sucrose in a concentration of 50-600 mg/mL, or 100-600 mg/mL, or 100-450 mg/mL, or 150-450 mg/mL, or 150-300 mg/mL.

13. The formulation according to claim 1, wherein the formulation contains sorbitol in a concentration of at least 400 mM.

14. The formulation according to claim 13, wherein said sorbitol is present in a concentration of 100-800 mg/mL, or 100-650 mg/mL, or 150-650 mg/mL, or 150-500 mg/mL, or 150-250 mg/mL.

15. The formulation according to claim 1, wherein the calculated osmotic concentration of the formulation is at most 1500 mOsm/L, 1200 mOsm/L, 1000 mOsm/L, or 900 mOsm/L.

16. The formulation according to claim 1, having a pH from 5.5-7.5, or from 6.0 to 7.0, or from 6.3 to 6.7.

17. The formulation according to claim 1, wherein the Factor VIII molecule is a recombinant full length FVIII or a recombinant B-domain truncated FVIII.

18. The formulation according to claim 1, wherein the Factor VIII molecule is a FVIII derivative or a FVIII analogue

19. The formulation according to claim 18, wherein the Factor VIII molecule is a pegylated FVIII, or a FVIII fusion protein, such as an albumin-fused FVIII, or an Fc region-fused FVIII.

20. The formulation according to claim 19, wherein the Factor VIII molecule is a glycopegylated B-domain truncated FVIII.

21. The formulation according to claim 1, wherein the FVIII molecule is a two-chain B-domain truncated FVIII molecule consisting of a heavy chain-Linker sequence (A1-a1-A2-a2-L) and a light chain sequence (a3-A3-C1-C2) held together by non-covalent interactions, wherein the Linker (L) is a 20 amino acid residue linker sequence (SFSQNSRHPSQNPPVLKRHQ) (SEQ ID NO 3); the heavy chain (A1-a1-A2-a2) and the light chain (a3-A3-C1-C2) correspond to the sequences as set forth in amino acid numbers 1-740 and 1649-2332, respectively, of SEQ ID NO: 1.

22. The formulation according to claim 1, wherein the FVIII molecule is a two-chain B-domain truncated FVIII molecule consisting of a heavy chain-Linker sequence (A1-a1-A2-a2-L) and a light chain sequence (a3-A3-C1-C2) held together by non-covalent interactions, wherein the Linker (L) is a 20 amino acid residue linker sequence (SFSQNSRHPSQNPPVLKRHQ) (SEQ ID NO 3); the heavy chain (A1-a 1-A2-a2) and the light chain (a3-A3-C1-C2) correspond to the sequences as set forth in amino acid numbers 1-740 and 1649-2332, respectively, of SEQ ID NO: 1, wherein one or more PEG group(s) has/have been attached to the FVIII polypeptide via a glycan located within the linker sequence (SEQ ID 3).

23. A method for optimising a liquid formulation of coagulation Factor VIII, the method comprising:
(i) providing one or more liquid formulations comprising Factor VIII to be tested;
(ii) adding a protein denaturant to said liquid formulations, and incubating the resulting solutions for a predetermined period of time;
(iii) analysing the incubated solutions of (ii) for the presence of dissociated Factor VIII; and
(iv) selecting one or more formulation(s) having a desired low level of dissociated Factor VIII.

24. A method for identifying a stable liquid formulation of Factor VIII, the method comprising:
(i) providing one or more liquid formulations comprising Factor VIII to be tested;
(ii) adding a protein denaturant to said liquid formulations, and incubating the resulting solutions for a predetermined period of time;
(iii) analysing the incubated solutions of (ii) for the presence of dissociated Factor VIII; and
(iv) selecting one or more formulation(s) having a desired low level of dissociated Factor VIII.

25. The method according to claim 23, wherein the protein denaturant is guanidinium chloride or urea.

26. The method according to claim 23, wherein the Factor VIII molecule is a recombinant full length FVIII or a recombinant B-domain truncated FVIII.

27. The method according to claim 23, wherein the Factor VIII molecule is a FVIII derivative or a FVIII analogue.

28. The method according to claim 26, wherein the Factor VIII polypeptide is a glycopegylated B-domain truncated FVIII.
